(19) United States
(12) Patent Application Publication
(10) Pub. No.: US 2008/0267949 A1
(43) Pub. Date: Oct. 30, 2008

(54) Peptides capable of binding to serum proteins

(75) Inventors: Hilde Adi Pierrette Revets, Meise (BE); Joost Alexander Kolkman, Sint-Martens-Latem (BE); Hendricus Renerus Jacobus Mattheus Hoogenboom, Maastricht (NL)

Correspondence Address: WOLF GREENFIELD & SACKS, P.C., 600 ATLANTIC AVENUE, BOSTON, MA, 02210-2206, US

(73) Assignee: Ablynx N.V., Ghent-Zwijnaarde (BE)

(21) Appl. No.: 11/999,497

(22) Filed: Dec. 5, 2007

Related U.S. Application Data
(60) Provisional application No. 60/872,923, filed on Dec. 5, 2006.

(51) Int. Cl. A61K 39/395; C07K 14/435; C07H 21/00; C12N 5/10; A61K 38/16; C40B 30/04; A61P 43/00

(52) U.S. Cl. 424/130.1; 530/350; 536/23.1; 435/325; 514/12; 506/9

(57) ABSTRACT

The present invention relates to amino acid sequences that are capable of binding to serum proteins; to compounds, proteins, polypeptides, fusion proteins or constructs comprising or essentially consisting of such amino acid sequences; to nucleic acids that encode such amino acid sequences, compounds, proteins, polypeptides, fusion proteins or constructs; to compositions, and in particular pharmaceutical compositions, that comprise such amino acid sequences, compounds, proteins, polypeptides, fusion proteins or constructs; and to uses of such amino acid sequences, compounds, proteins, polypeptides, fusion proteins or constructs.

### RELATED APPLICATIONS

This application claims the benefit under 35 U.S.C. § 119(e) of U.S. provisional application Ser. No. 60/872,923 filed on Dec. 5, 2006, the entire disclosure of which is incorporated herein by reference.

### FIELD OF THE INVENTION

The present invention relates to amino acid sequences that are capable of binding to serum proteins; to compounds, proteins, polypeptides, fusion proteins or constructs comprising or essentially consisting of such amino acid sequences; to nucleic acids that encode such amino acid sequences, compounds, proteins, polypeptides, fusion proteins or constructs; to compositions, and in particular pharmaceutical compositions, that comprise such amino acid sequences, compounds, proteins, polypeptides, fusion proteins or constructs; and to uses of such amino acid sequences, compounds, proteins, polypeptides, fusion proteins or constructs.

Other aspects, embodiments, advantages and applications of the invention will become clear from the further description herein.

### BACKGROUND OF THE INVENTION

Amino acid sequences that are capable of binding to serum proteins and uses thereof in compounds, proteins, polypeptides, fusion proteins or constructs in order to increase the half-life of therapeutically relevant proteins, polypeptides and other compounds are known in the art.

For example, WO 91/01743, WO 01/45746 and WO 02/076489 describe peptide moieties binding to serum albumin that can be fused to therapeutic proteins and other therapeutic compounds and entities in order to increase the half-life thereof. However, these peptide moieties are of bacterial or synthetic origin, which is less preferred for use in therapeutics.

The neonatal Fc receptor (FcRn), also termed “Brambell receptor”, is involved in prolonging the life-span of albumin in circulation (see Chaudhury et al., The Journal of Experimental Medicine, vol. 3, no. 197, 315-322 (2003)). The FcRn receptor is an integral membrane glycoprotein consisting of a soluble light chain consisting of β2-microglobulin, noncovalently bound to a 43 kD α chain with three extracellular domains, a transmembrane region and a cytoplasmic tail of about 50 amino acids. The cytoplasmic tail contains a dinucleotide motif-based endocytosis signal implicated in the internalization of the receptor. The α chain is a member of the nonclassical MHC I family of proteins. The β2m association with the α chain is critical for correct folding of FcRn and exiting the endoplasmic reticulum for routing to endosomes and the cell surface.

The overall structure of FcRn is similar to that of class I molecules. The α-1 and α-2 regions resemble a platform composed of eight antiparallel β strands forming a single β-sheet topped by two antiparallel α-helices very closely resembling the peptide cleft in MHC 1 molecules. Owing to an overall repositioning of the α-1 helix and bending of the C-terminal portion of the α-2 helix due to a break in the helix introduced by the presence of Pro 162, the FcRn helices are considerably closer together, occluding peptide binding. The side chain of Arg164 of FcRn also occludes the potential interaction of the peptide N-terminus with the MHC pocket. Further, salt bridge and hydrophobic interaction between the α-1 and α-2 helices may also contribute to the groove closure.

FcRn therefore, does not participate in antigen presentation, and the peptide cleft is empty.

FcRn binds and transports IgG across the placental syncytiotrophoblast from maternal circulation to fetal circulation and protects IgG from degradation in adults. In addition to homeostasis, FcRn controls transcytosis of IgG in tissues. FcRn is localized in epithelial cells, endothelial cells and hepatocytes.

According to Chaudhury et al. (supra), albumin binds FcRn to form a tri-molecular complex with IgG. Both albumin and IgG bind noncooperatively to distinct sites on FcRn. Binding of human FcRn to Sepharose-HSA and Sepharose-hIgG was pH dependent, being maximal at pH 5.0 and nil at pH 7.0 through pH 8. The observation that FcRn binds albumin in the same pH dependent fashion as it binds IgG suggests that the mechanism by which albumin interacts with FcRn and thus is protected from degradation is identical to that of IgG, and mediated via a similarly pH-sensitive interaction with FcRn. Using SPR to measure the capacity of individual HSA domains to bind immobilized soluble hFcRn, Chaudhury showed that FcRn and albumin interact via the D-III domain of albumin in a pH-dependent manner, on a site distinct from the IgG binding site (Chaudhury, PhD dissertation, see http://www.andersonlab.com/biosketchCC.htm; Chaudhury et al. Biochemistry, ASAP Article 10.1021/bi052628y S0006-2960(05)02628-0 (Web release date: Mar. 22, 2006)).

WO 04/041865 by Ablynx N.V. describes Nanobodies® capable of binding to serum albumin (and in particular against human serum albumin) that can be linked to other proteins (such as one or more other Nanobodies® capable of binding to a desired target) in order to increase the half-life of said protein. It is known that these Nanobodies® are more potent and more stable than conventional four-chain serum albumin binding antibodies which leads to (1) lower dosage forms, less frequent dosage leading to less side effects; (2) improved stability leading to a broader choice of administration routes, comprising oral or subcutaneous routes in addition to the intravenous route; (3) lower treatment cost due to lower cost of goods.

Notwithstanding the foregoing, there remains a need for alternative techniques and moieties that can be used to increase the half-life of therapeutically relevant proteins, polypeptides and (other) compounds. For example, some of the peptide moieties described in the art are from synthetic or semi-synthetic origin, and may therefore contain undesired epitopes that may be recognized by the human immune system, which may give rise to immunogenic properties. Also, serum protein binding peptides that are smaller than the serum protein binding domain antibodies and Nanobodies® described in the art (which may sometimes have a higher molecular weight than the compound to which they are to be fused or linked) may be easier to handle, to fuse or to link to a therapeutic protein, polypeptide or compound, and/or to express as (part of) a recombinant (fusion) polypeptide; may have superior biophysical properties (such as solubility, stability); and in fusions or constructs in which they are linked to a therapeutic protein, polypeptide or compound, may result in reduced steric hindrance or other undesired interactions with the fusion partner or its desired pharmacological properties.

WO 03/050531 (Ablynx N.V. and Algonomics N.V.) describes methods for the identification and selection of peptides, in particular immunoglobulin heavy chain variable domain CDR sequences that bind to a given target or targets of interest. It is shown that especially CDR3 plays a crucial role in antigen binding (Kabat and Wu, 1991) and a number of cases have been reported where CDR3 peptides show antigen binding mimicking the parental antibody (reference is for example made to Taub et al., 1991). For Nanobodies the dominant role of the CDR3 in the antigen binding interaction is even more apparent (De Genst et al., 2006).

### SUMMARY OF THE INVENTION

It is an object of the present invention to provide amino acid sequences that are an alternative, and in particular an improved alternative, to the serum protein-binding amino acid sequences described in the prior art cited above.

Generally, the invention achieves this objective by providing amino acid sequences that can bind to serum proteins and that can be used as small peptides or as peptide moieties for linking or fusing to a therapeutic compound (such as a protein or polypeptide) in order to increase the half-life thereof. These amino acid sequences (which are also referred to herein as “amino acid sequences of the invention”) as further defined herein.

Thus, according to a first aspect, the invention relates to an amino acid sequence that can bind to a serum protein and that essentially consists of a CDR sequence (and in particular, a single CDR sequence).

Said amino acid sequence preferably has a length of less than 90 amino acid residues, preferably less than 50 amino acid residues, such as about 40, 30 or 20 amino acid residues; and/or is preferably such that it does not contain an immunoglobulin from and is also not capable of forming an immunoglobulin fold.

The amino acid sequences of the invention preferably contain a CDR sequence (and in particular, a single CDR sequence), and in particular a CDR sequence that is such that it can bind to a serum protein, so as to enable the amino acid sequence to bind to the serum protein.

The CDR sequence may in particular be a CDR sequence that has been derived from an immunoglobulin variable domain that can bind to a serum protein. The CDR sequence may also essentially consists of a fragment of an immunoglobulin variable domain that comprises a CDR sequence.

More in particular, the CDR sequence may be derived from an immunoglobulin variable domain, which is selected from the group consisting of a V_{H}-domain, a V_{L}-domain, a V_{HH}-domain or an antigen-binding fragment of an immunoglobulin variable domain; and/or may be a fragment of a V_{H}-domain, a V_{L}-domain, a V_{HH}-domain or an antigen-binding fragment of an immunoglobulin variable domain that comprises a CDR sequence.

Preferably, the CDR sequence is derived from an immunoglobulin variable domain, which is selected from the group consisting of a human variable domain, a (single) domain antibody, a dAb, or a Nanobody®; and/or is a fragment of a human variable domain, a (single) domain antibody, a dAb, or a Nanobody®. CDR sequences derived from Nanobodies are particularly preferred.

The CDR sequence preferably has a length between 3 and 40 amino acid residues, preferably between 5 and 30 amino acid residues. In particular, the CDR sequence may be a CDR2 sequence or a CDR3 sequence.

The amino acid sequence of the invention is preferably such that it binds to a serum protein in such a way that the half-life of the serum protein molecule is not (significantly) reduced.

The serum protein to which the amino acid sequence of the invention binds may in particular be a serum protein chosen from the group consisting of serum albumin, serum immunoglobulins such as IgG, thyroxine-binding protein, transferrin, fibrinogen. The amino acid sequence of the invention may also bind to at least one part, fragment, epitope or domain of any of the foregoing.

Preferably, the amino acid sequence of the invention binds to serum albumin or at least one part, fragment, epitope or domain thereof; and in particular to human serum albumin or at least one part, fragment, epitope or domain thereof. When the amino acid sequence of the invention binds to (human) serum albumin, it preferably is capable of binding to amino acid residues on serum albumin that are not involved in binding of (human) serum albumin to FcRn; and/or of binding to amino acid residues on serum albumin that do not form part of domain III of (human) serum albumin. Reference is made to WO 06/0122787.

The amino acid sequence of the invention preferably comprises a CDR sequence flanked by two flanking amino acid sequences on either side of the CDR sequence. Said two flanking amino acid sequences preferably each have a length of between 1 and 30 amino acid residues, preferably between 2 and 20 amino acid residues, such as about 5, 10 or 15 amino acid residues; and may in particular be derived from immunoglobulin framework sequences and/or may be fragments of immunoglobulin framework sequences. More in particular, said two flanking amino acid sequences may be immunoglobulin framework sequences that have been derived from the framework sequences that, in the immunoglobulin variable domain from which said CDR sequence is derived, are adjacent to said CDR sequence; and/or may be are fragments of the framework sequences that, in the immunoglobulin variable domain from which said CDR sequence is derived, are adjacent to said CDR sequence.

For example, when the CDR sequence is a CDR2 sequence, the flanking sequences are preferably immunoglobulin framework sequences that have been derived from a framework 2 sequence and a framework 3 sequence, respectively; and/or fragments of a framework 2 sequence and a framework 3 sequence, respectively. When the CDR sequence is a CDR3 sequence, the flanking sequences are preferably immunoglobulin framework sequences that have been derived from a framework 3 sequence and a framework 4 sequence, respectively; and/or fragments of a framework 3 sequence and a framework 4 sequence, respectively.

In one particularly preferred embodiment, the amino acid sequences of the invention contain at least two cysteine residues that are capable of forming a disulphide bridge, and/or that form part of an intramolecular disulphide bridge. Preferably, said cysteine residues are located in the flanking amino acid sequences. For example, when the flanking amino acid sequences are derived from immunoglobulin framework sequences and/or are fragments or immunoglobulin framework sequences, said cysteine residues may be cysteine residues that naturally occur in said immunoglobulin framework sequences and/or cysteine residues that have been introduced into said in immunoglobulin framework sequences.

In one specific, but non-limiting aspect of the invention, the amino acid sequence of the invention is in a “constrained” format (i.e. comprising at least one disulphide bridge that links the flanking sequences) or is an amino acid sequence that is capable of binding (as described herein) to human serum albumin when it is in a constrained format. In particular, such an amino acid sequence comprises a CDR sequence that is such that, when the amino acid sequence is in a constrained format, is capable of binding (as described herein) to human serum albumin.

In another specific, but non-limiting aspect of the invention, the amino acid sequence of the invention is in a “non-constrained” format (i.e. not comprising any disulphide bridge that links the flanking sequences) or is an amino acid sequence that is capable of binding (as described herein) to human serum albumin when it is in a non-constrained format. In particular, such an amino acid sequence comprises a CDR sequence that is such that, when the amino acid sequence is in a non-constrained format, is capable of binding (as described herein) to human serum albumin.

In yet another specific, but non-limiting aspect of the invention, the amino acid sequence of the invention is an amino acid sequence that is capable of binding (as described herein) to human serum albumin when it is in both a constrained format as well as a non-constrained format. Such an amino acid sequence may be in both a constrained format as well as in a non-constrained format. In particular, such an amino acid sequence comprises a CDR sequence that is such that, when the amino acid sequence is in either a constrained format or a non-constrained format, is capable of binding (as described herein) to human serum albumin.

A non-limiting example of an amino acid sequence of the invention is given in SEQ ID NO:1, with the corresponding nucleotide sequence being given in SEQ ID NO:2. This amino acid sequence DTAVYYCNAAASYSDYDVFGGGTDFGPWGQGTQV (SEQ ID NO:1) comprises a CDR sequence AASYSDYDVFGGGTDFGP (SEQ ID NO:3) flanked by two framework sequences (indicated in italics), which are derived from framework 3 and 4, respectively. This CDR sequence can bind to serum albumin in the form of the amino acid sequence when it is in the form of the peptide of SEQ ID NO:1, but also as such (i.e. without the flanking FR sequences). Reference is made to Example 4 below, which shows that this CDR sequence can bind to human serum albumin in both a constrained format (i.e. containing a disulphide bridge, see for example the peptide 17D12-CDR3-C, SEQ ID NO: 27) as well as in a non-constrained format (i.e. without a disulphide bridge, see for example the peptide 17D12-CDR3-NC, SEQ ID NO: 26).

Thus, in one preferred, but non-limiting aspect, the amino acid sequence of the invention is an amino acid sequence that at least comprises the amino acid sequence AASYSDYDVFGGGTDFGP (SEQ ID NO:3) or that comprises an amino acid sequence that differs from the amino acid sequence AASYSDYDVFGGGTDFGP (SEQ ID NO:3) by no more than 9 amino acid differences (as defined herein), preferably no more than 6 amino acid differences, such as 5, 4, 3, 2 or only 1 amino acid difference. Such amino acid sequences may be as further described herein.

For example, such an amino acid sequence of the invention may comprise the amino acid sequence AASYSDYDVFGGGTDFGP (SEQ ID NO:3) (or an amino acid sequence that differs from this sequence by no more than 9 amino acid differences, preferably no more than 6 amino acid differences, such as 5, 4, 3, 2 or only 1 amino acid difference), and may further comprise one or two flanking amino acid sequences (i.e. at either end or both ends of the sequence, respectively). Also, such an amino acid sequence of the invention may be in a constrained or a non-constrained format.

Preferably, such an amino acid sequence of the invention (or a compound of the invention comprising at least one such amino acid sequence, as further described herein) is such that it can bind to a serum albumin, and in particular to human serum albumin:
with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
with a kₒₙ-rate of between 10² M⁻¹s⁻¹ to about 10⁷ M⁻¹s⁻¹, preferably between 10⁷ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or
with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁴ s⁻¹, such as between 10⁻⁴ s⁻¹ and 10⁻⁶ s⁻¹;
   and such amino acid sequences (and nucleotide sequences encoding the same, as well as compounds of the invention comprising the same) form further aspects of the invention.

Preferably, such an amino acid sequence of the invention (or a compound of the invention comprising one such amino acid sequence, as further described herein) is such that it will bind to the serum protein with an affinity less than 500 nM, preferably less than 200 nM, more preferably less than 10 nM, such as less than 500 μM.

When such an amino acid sequence is an amino acid sequence that differs from the amino acid sequence AASYSDYDVFGGGTDFGP (SEQ ID NO:3) by no more than 9 amino acid differences (and preferably by no more than 6 amino acid differences, such as by 5, 4, 3, 2 or only 1 amino acid difference), it is preferably such that it (or a compound of the invention comprising at least one such amino acid sequence, as further described herein) can bind to a serum albumin, and in particular to human serum albumin, with a K_{D}, K_{A}, Kₒₙ and/or K_{off} that is as mentioned in the preceding paragraph. such amino acid sequences (and nucleotide sequences encoding the same, as well as compounds of the invention comprising the same, as further described herein) form a further aspect of the invention. For example, such an amino acid sequence may be an amino acid sequence that has been obtained by affinity maturation starting from the amino acid sequence AASYSDYDVFGGGTDFGP (SEQ ID NO:3).

When such an amino acid sequence is an amino acid sequence that differs from the amino acid sequence AASYSDYDVFGGGTDFGP (SEQ ID NO:3) by no more than 9 amino acid differences (and preferably by no more than 6 amino acid differences, such as by 5, 4, 3, 2 or only 1 amino acid difference), it may comprise a total of between 9 and 27 amino acid residues, such as between 12 and 24 amino acid residues, for example between 15 and 21 amino acid residues, such as 16, 17, 18, 19 or 20 amino acid residues). Again, such an amino acid sequence is preferably such that it can bind to a serum albumin, and in particular to human serum albumin, with a K_{D}, K_{A}, Kₒₙ and/or K_{off} that is as mentioned in the preceding paragraph; and such amino acid sequences (and nucleotide sequences encoding the same, as well as compounds of the invention comprising the same, as further described herein) form a further aspect of the invention. For example, such an amino acid sequence may be an amino acid sequence that has been obtained by affinity maturation starting from the amino acid sequence AASYSDYDVFGGGTDFGP (SEQ ID NO:3).

Also, preferably, when such an amino acid sequence is an amino acid sequence that differs from the amino acid sequence AASYSDYDVFGGGTDFGP (SEQ ID NO:3) by no more than 9 amino acid differences (and preferably by no more than 6 amino acid differences, such as by 5, 4, 3, 2 or only 1 amino acid difference), it is preferably such that it comprises one or more (such as one, two, three, four or five) stretches of amino acid residues that comprise at least 3 (such as at least 4, 5, 6, 7, 8, 9 or more) contiguous amino acid residues from the sequence AASYSDYDVFGGGTDFGP (SEQ ID NO:3) (again such that the total number of amino acid residues is between 9 and 27 amino acid residues, such as between 12 and 24 amino acid residues, for example between 15 and 21 amino acid residues, such as 16, 17, 18, 19 or 20 amino acid residues). Again, such an amino acid sequence is preferably such that it can bind to a serum albumin, and in particular to human serum albumin, with a K_{D}, K_{A}, Kₒₙ and/or K_{off} that is as mentioned in the preceding paragraphs; and such amino acid sequences (and nucleotide sequences encoding the same, as well as compounds of the invention comprising the same, as further described herein) form a further aspect of the invention. For example, such an amino acid sequence may be an amino acid sequence that has been obtained by affinity maturation starting from the amino acid sequence AASYSDYDVFGGGTDFGP (SEQ ID NO:3).

In one specific, but not-limiting aspect, such an amino acid sequence is an amino acid sequence that (i) differs from the amino acid sequence AASYSDYDVFGGGTDFGP (SEQ ID NO:3) by no more than 9 amino acid differences (and preferably by no more than 6 amino acid differences, such as by 5, 4, 3, 2 or only 1 amino acid difference); (ii) has been obtained by affinity maturation starting from the amino acid sequence AASYSDYDVFGGGTDFGP (SEQ ID NO:3); (iii) comprises a total of between 9 and 27 amino acid residues, such as between 12 and 24 amino acid residues, for example between 15 and 21 amino acid residues, such as 16, 17, 18, 19 or 20 amino acid residues), and preferably comprises one or more (such as one, two, three, four or five) stretches of amino acid residues that comprise at least 3 (such as at least 4, 5, 6, 7, 8, 9 or more) contiguous amino acid residues from the sequence AASYSDYDVFGGGTDFGP (SEQ ID NO:3); and (iv) it can bind to a serum albumin, and in particular to human serum albumin, with a K_{D}, K_{A}, Kₒₙ and/or K_{off} that is as mentioned in the preceding paragraphs.

Again, all the above amino acid sequences may be as further described herein, and may for example comprise one or two flanking amino acid sequences (i.e. at either end or both ends of the sequence, respectively), and may be in a constrained or a non-constrained format. For example, such amino acid sequences may further be such that they are capable of binding (as described herein) to a serum albumin, and in particular to human serum albumin, in a constrained format, in a non-constrained format, and preferably in both a constrained and non-constrained format.

Also, compounds of the invention that comprise one or more of the above amino acid sequences form a further specific aspect of the invention, and such compounds of the invention may be as further described herein (and are preferably in accordance with the preferred aspects described herein for compounds of the invention).

The invention also relates to a compound or construct which comprises at least one amino acid sequence of the invention and at least one therapeutic moiety (also referred to herein as “compounds of the invention”). Again, the amino acid sequence(s) of the invention present in such a compound or construct preferably contain at least two cysteine residues that are capable of forming a disulphide bridge, and/or that form part of an intramolecular disulphide bridge (for example, and in particular, in the two flanking sequences that flank the CDR sequence).

For example, and without limitation, a compound of the invention may comprise the at least one therapeutic moiety, that is linked to one, two, three, four or more amino acid sequences of the invention. For example, when the therapeutic moiety is a protein or polypeptide, the one or more amino acid sequences of the invention may be linked to the C-terminus of the protein or polypeptide (either directly or via a suitable spacer or linker); to the N-terminus of the protein or polypeptide (again either directly or via a suitable spacer or linker); or both to the C-terminus and the N-terminus. When a compound of the invention comprises two or more amino acid sequences of the invention, these may be the same or different.

The therapeutic moiety may also be linked (either at its C-terminus, its N-terminus, or both, and again either directly or via a suitable spacer or linker) to a concatamer that comprises at least two (such as two, three or four) amino acid sequences of the invention (which may be the same or different), that may either be linked directly to each other, or via a suitable linker or spacer. Such (bivalent, trivalent or multivalent) concatamers (and nucleotide sequences encoding the same, as well as compounds of the invention comprising the same) form a further aspect of the invention, and may bind to serum albumin with a higher avidity than a monomeric amino acid sequence of the invention.

Also, when a compound of the invention comprises two or more therapeutic moieties, each of these therapeutic moieties (or both) may be linked to one or more amino acid sequences of the invention, as further described herein. Also, the two or more therapeutic moieties may be linked to each other via a linker that comprises or essentially consists of one or more amino acid sequences of the invention (and optionally further linking amino acid sequences), and such a linker (as well as compounds of the invention comprising the same) form a further aspect of the invention.

The at least one therapeutic moiety preferably comprises or essentially consists of an amino acid sequence, and in particular may comprise or essentially consist of an immunoglobulin sequence or an antigen-binding fragment thereof (for example, an antibody or an antigen-binding fragment thereof), such as an immunoglobulin variable domain or an antigen-binding fragment thereof (for example, a V_{H}-domain, a V_{L}-domain, a V_{HH}-domain or an antigen-binding fragment thereof); or a protein or polypeptide comprising the same (for example, an scFv construct). For such constructs, reference is for example made to the review by Holliger and Hudson, Nat. Biotechnol. 2005 September; 23(9):1126-36 and the further prior art cited therein.

According to one specific, but non-limiting aspect, the therapeutic moiety comprises or essentially consists of a (single) domain antibody, a “dAb”, or a Nanobody®.

In a compound of the invention the one or more amino acid sequences of the invention may be either directly linked to the at least one therapeutic moiety or linked to the at least one therapeutic moiety via one or more suitable linkers or spacers. Suitable linkers will be clear to the skilled person, for example based on the further disclosure herein. When the one or more therapeutic moieties are amino acid sequences, the linkers or spacers preferably comprise or essentially consist of amino acid sequences, so that the resulting compound or construct essentially consists of a (fusion) protein or (fusion) polypeptide (also referred to herein as a “polypeptide of the invention”). Again, the amino acid sequence(s) of the invention present in such a polypeptide of the invention preferably contain at least two cysteine residues that are capable of forming a disulphide bridge, and/or that form part of an intramolecular disulphide bridge (for example, and in particular, in the two flanking sequences that flank the CDR sequence).

The invention also relates to a nucleotide sequence or nucleic acid that encodes an amino acid sequence of the invention or a polypeptide of the invention (also referred to herein as a “nucleotide sequence of the invention” or a “nucleic acid of the invention”). Again, such a nucleic acid of the invention preferably encodes an amino acid sequence of the invention or a polypeptide of the invention that contains at least two cysteine residues that are capable of forming a disulphide bridge (for example, and in particular, in the two flanking sequences that flank the CDR sequence).

The invention also relates to a host or host cell that contains a nucleotide sequence or nucleic acid of the invention and/or that expresses (or is capable of expressing) an amino acid sequence of the invention or a polypeptide of the invention, and in particular an amino acid sequence of the invention or a polypeptide of the invention that contains at least two cysteine residues that are capable of forming a disulphide bridge (for example, and in particular, in the two flanking sequences that flank the CDR sequence).

As will be clear to the skilled person based on the disclosure herein, one preferred but non-limiting aspect of the invention relates to amino acid sequences of the invention that contain a disulphide bridge, in particular between (the cysteine residues present in each of) the two flanking sequences that flank the CDR sequence. Accordingly, the invention also relates to a method for preparing such an amino acid sequence of the invention, which method generally comprises at least the step of forming a disulphide bridge in an amino acid sequence of the invention that comprises at least two cysteine residues that are capable of forming a disulphide bridge, and in particular at least the step of forming a disulphide bridge between (the cysteine residues present in each of) the two flanking sequences that flank the CDR sequence.

The invention also relates to polypeptides of the invention that contain a disulphide bridge, in particular in the part of the polypeptide that is composed of the amino acid sequence of the invention. Accordingly, the invention also relates to a method for preparing such a polypeptide of the invention, which method generally comprises at least the step of forming a disulphide bridge in a polypeptide of the invention, in particular in the part that is formed by the amino acid sequence of the invention. Again, for this purposes, the amino acid sequence of the invention present in the polypeptide preferably comprises at least two cysteine residues that are capable of forming a disulphide bridge, in particular in each of the two flanking sequences that flank the CDR sequence.

Another method for preparing the amino acid sequences or polypeptides of the invention generally comprises at least the step of:
a) expressing a nucleotide sequence or nucleic acid of the invention; and optionally further comprises:
b) isolating the amino acid sequence of the invention or the polypeptide of the invention, respectively, so expressed.

Where the amino acid sequence of the invention or the polypeptide of the invention so obtained contains at least two cysteine residues that are capable of forming a disulphide bridge (for example, and in particular, in the two flanking sequences that flank the CDR sequence), said method may also comprise a further step of forming such a disulphide bridge, as further described herein.

Yet another method for preparing the amino acid sequences or polypeptides of the invention generally comprises at least the step of:
a) cultivating or maintaining a host or host cell as described herein under conditions such that said host or host cell produces an amino acid sequence or polypeptide of the invention;
   and optionally further comprising:
b) isolating the amino acid sequence of the invention or polypeptide of the invention respectively, thus obtained.

Again, where the amino acid sequence of the invention or the polypeptide of the invention so obtained contains at least two cysteine residues that are capable of forming a disulphide bridge (for example, and in particular, in the two flanking sequences that flank the CDR sequence), said method may also comprise a further step of forming such a disulphide bridge, as further described herein.

The invention also relates to the amino acid sequences, compounds, construct or polypeptides obtained via the above methods.

The invention further relates to a pharmaceutical composition that comprises at least one amino acid sequence, compound, construct or polypeptide as described herein; and optionally at least one pharmaceutically acceptable carrier, diluent or excipient.

The invention also relates to some specific methods for providing amino acid sequences (such as CDR sequences) that can bind to a serum protein and that can be used in the present invention (i.e. as an amino acid sequence of the invention or as a starting point for providing an amino acid sequence of the invention). One such specific method at least comprises the steps of:
a) providing a set, collection or library of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold;
b) screening said set, collection or library for amino acid sequences that can bind to and/or have affinity for a serum protein or at least one part, fragment, epitope or domain thereof; and
c) isolating the amino acid sequence(s) that can bind to and/or have affinity for said serum protein or said at least one part, fragment, epitope or domain thereof.

In step b) of such a method, said set, collection or library of amino acid sequences is preferably screened for amino acid sequences that can bind to and/or have affinity for a serum protein chosen from the group consisting of serum albumin, serum immunoglobulins such as IgG, thyroxine-binding protein, transferring or fibrinogen; and/or for amino acid sequences that can bind to and/or have affinity for at least one part, fragment, epitope or domain of serum albumin, serum immunoglobulins such as IgG, thyroxine-binding protein, transferrin or fibrinogen.

In particular, in step b) of such a method, said set, collection or library of amino acid sequences may be screened for amino acid sequences that can bind to and/or have affinity for serum albumin or at least one part, fragment, epitope or domain thereof; and more in particular for amino acid sequences that can bind to and/or have affinity for human serum albumin or at least one part, fragment, epitope or domain thereof. According to one specific, but non-limiting aspect, in step b) of such a method, said set, collection or library of amino acid sequences may be screened for one or more amino acid sequences that can bind to and/or have affinity for a part, fragment, epitope or domain of (human) serum albumin that is not involved in binding of (human) serum albumin to FcRn; and/or for amino acid sequences that can bind to and/or have affinity for at least one part, fragment, epitope or domain of (human) serum albumin that does not form part of domain III of (human) serum albumin.

Said screening may be performed in any manner for protein screening known per se. For example, the set, collection or library of amino acid sequences may be displayed on a phage, phagemid, ribosome or suitable micro-organism using techniques known to the skilled person. Reference is for example made to the review by Hoogenboom et al, Nat Biotechnol 23:1105, 2005 and the further prior art cited therein.

The set, collection or library of amino acid sequences used in the above method preferably comprises a set, collection or library of amino acid sequences that essentially consist of a CDR sequence flanked by two flanking amino acid sequences that have been derived from the immunoglobulin framework sequences; and/or a set, collection or library of fragments of immunoglobulin sequences that comprise a CDR sequence flanked on both sides by framework sequences or fragments of framework sequences. In particular, the set, collection or library of amino acid sequences may comprise a set, collection or library of amino acid sequences that comprise or essentially consist of a CDR sequence flanked by two flanking amino acid sequences that have been derived from the framework sequences that, in the immunoglobulin variable domain from which said CDR sequence is derived, are adjacent to said CDR sequence. For example, the set, collection or library of amino acid sequences may comprise or essentially consist of CDR2 sequences flanked by two flanking amino acid sequences that have been derived from a framework 2 sequence and a framework 3 sequence, respectively; or of CDR3 sequences flanked by two flanking amino acid sequences that have been derived from a framework 3 sequence and a framework 4 sequence, respectively.

For providing amino acid sequences that contain two cysteine residues for forming a disulphide bridge (as further described herein), the above method may further comprise introducing (i.e. by adding, inserting or substituting) of one or two cysteine residues, such that each framework sequence in the resulting amino acid sequence contains at least one cysteine residue.

Alternatively, where the amino acid sequence thus obtained does not already comprise flanking amino acid sequences, such flanking sequences (preferably again with cysteine residues) may be added.

The set, collection or library of amino acid sequences that is used in step a) of the above method (and that is subsequently screened in step b)) may be any suitable set, collection or library of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold. For example, it may be a set, collection or library of amino acid sequences that has been obtained by method that comprises the use of one or more techniques for affinity maturation known per se.

However, according to one preferred aspect, such a set, collection or library may be obtained by a method that at least comprises the steps of:
a) providing a set, collection or library of nucleotide sequences that encode immunoglobulin sequences;
b) amplifying said nucleotide sequences using a combination of site-specific primers, such that the amplified fragments encode a set, library or collection of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold;
c) expressing the amplified fragments obtained in step b), so as to provide a set, library or collection of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold.

The set, collection or library of nucleotide sequences that encode immunoglobulin sequences that is used in step a) of the above method is may be any suitable set, collection or library of nucleotide sequences that encode immunoglobulin sequences (as generally understood by a person skilled in the art, for example an antibody, a variable domain of an antibody, or a fragment of an antibody comprising a variable domain), but may in particular be an immune set, collection or library, and more in particular an immune set, collection or library that has been obtained from mammal that has been suitably immunized with a serum protein (i.e. so as to raise an immune response against said serum protein). This set, collection or library may be generated in any manner known per se, such as by repertoire cloning (see for example WO 90/05144 or the review by Hoogenboom cited herein).

In one specific, but non-limiting aspect, said set, collection or library of nucleotide sequences that encode immunoglobulin sequences may be an immune set, collection or library of nucleotide sequences that encode heavy chain antibodies or VHH sequences, that have been obtained from a Camelid that has been suitably immunized with serum protein (i.e. so as to raise an immune response against said serum protein). For this, reference is for example made to the prior art cited herein.

The amplification step b) is preferably performed using (a combination of) site-specific primers that are specific for and/or capable of hybridizing to (i.e. under the conditions used for the amplification) nucleotide sequences that encode the framework sequences that flank said CDR sequence. For example, said (combination of) site-specific primers may be such that the amplified fragments encode a set, library or collection of amino acid sequences that (i) essentially consist of a CDR2 sequence; and/or (ii) comprise a fragment of an immunoglobulin that comprises a CDR2 sequences; and/or (iii) comprise a CDR2 sequence but that do not comprise an immunoglobulin fold and are also not capable of forming an immunoglobulin fold; in which case said site-specific primers may be specific for and/or capable of hybridizing to (i.e. under the conditions used for the amplification) nucleotide sequences that encode framework 2 sequences and framework 3 sequences, respectively.

Alternatively, said (combination of) site-specific primers may be such that the amplified fragments encode a set, library or collection of amino acid sequences that (i) essentially consist of a CDR3 sequence; and/or (ii) comprise a fragment of an immunoglobulin that comprises a CDR3 sequences; and/or (iii) comprise a CDR3 sequence but that do not comprise an immunoglobulin fold and are also not capable of forming an immunoglobulin fold; in which case said site-specific primers may be specific for and/or capable of hybridizing to (i.e. under the conditions used for the amplification) nucleotide sequences that encode framework 3 sequences and framework 4 sequences, respectively.

Another specific method for providing amino acid sequences (such as CDR sequences) that can bind to a serum protein and that can be used in the present invention (i.e. as an amino acid sequence of the invention or as a starting point for providing an amino acid sequence of the invention) may comprise the steps of:
a) providing a set, collection or library of immunoglobulin sequences;
b) screening said set, collection or library of immunoglobulin sequences for immunoglobulin sequences that can bind to and/or have affinity for a serum protein or at least one part, fragment, epitope or domain thereof;
c) determining the nucleotide sequence and/or the amino acid sequence of at least one immunoglobulin sequence that can bind to and/or has affinity for a serum protein or at least one part, fragment, epitope or domain thereof, as identified during step b); and/or determining the nucleotide sequence and/or the amino acid sequence of a CDR sequence thereof and/or of a fragment thereof that comprises a CDR sequence;
d) preparing, using any suitable technique known per se, an amino acid sequence according of the invention that (i) essentially consist of a CDR sequence with an amino acid sequence that has been determined in step c); and/or (ii) comprises a fragment of an immunoglobulin with an amino acid sequence that has been determined in step c); and/or (iii) comprises a CDR sequence with an amino acid sequence that has been determined in step c), but that does not comprise an immunoglobulin fold and are also not capable of forming an immunoglobulin fold.

Again, in step b) of this method, said set, collection or library of immunoglobulin sequences may be screened for immunoglobulin sequences that can bind to and/or have affinity for a serum protein chosen from the group consisting of serum albumin, serum immunoglobulins such as IgG, thyroxine-binding protein, transferring or fibrinogen; and/or for immunoglobulin sequences that can bind to and/or have affinity for at least one part, fragment, epitope or domain of serum albumin, serum immunoglobulins such as IgG, thyroxine-binding protein, transferrin or fibrinogen.

In particular, said set, collection or library of immunoglobulin sequences may be screened for immunoglobulin sequences that can bind to and/or have affinity for serum albumin or at least one part, fragment, epitope or domain thereof; and more in particular for human serum albumin or at least one part, fragment, epitope or domain thereof. According to one specific, but non-limiting aspect, in step b) of such a method, said set, collection or library of amino acid sequences may be screened for one or more amino acid sequences that can bind to and/or have affinity for a part, fragment, epitope or domain of (human) serum albumin that is not involved in binding of (human) serum albumin to FcRn; and/or for amino acid sequences that can bind to and/or have affinity for at least one part, fragment, epitope or domain of (human) serum albumin that does not form part of domain III of (human) serum albumin.

Again, said screening may be performed in any manner for protein screening known per se. For example, the set, collection or library of amino acid sequences may be displayed on a phage, phagemid, ribosome or suitable micro-organism using techniques known to the skilled person. Reference is for example made to the review by Hoogenboom et al, Nat Biotechnol 23:1105, 2005 and the further prior art cited therein.

The set, collection or library of immunoglobulin sequences used in step a) of the above method may be any suitable set, collection or library of immunoglobulin sequences, such as a naïve set, collection or library of immunoglobulin sequences, a synthetic or semi-synthetic set, collection or library of immunoglobulin sequences, or a set, collection or library of immunoglobulin sequences that have been subjected to affinity maturation. According to one specific, but non-limiting aspect, said set, collection or library of immunoglobulin sequences may be an immune set, collection or library of immunoglobulin sequences, and in particular an immune set, collection or library that has been obtained from mammal that has been suitably immunized with a serum protein (i.e. so as to raise an immune response against said serum protein). For example, the set, collection or library of immunoglobulin sequences used in step a) may be an immune set, collection or library of heavy chain antibodies or VHH sequences, that have been obtained from a Camelid that has been suitably immunized with serum protein (i.e. so as to raise an immune response against said serum protein). For methods of providing such a set, collection or library, reference is again made to the prior art cited herein.

The set, collection or library of immunoglobulin sequences is a preferably set, collection or library of CDR sequences derived from heavy chain variable domains or of light chain variable domains, and may in particular be a set, collection or library of domain antibodies, single domain antibodies or immunoglobulin sequences that are capable of functioning as a domain antibody or single domain antibody.

Also, in step c), preferably the sequence of a CDR2 sequence or a CDR3 sequence is determined.

Another specific method for providing amino acid sequences (such as CDR sequences) that can bind to a serum protein and that can be used in the present invention (i.e. as an amino acid sequence of the invention or as a starting point for providing an amino acid sequence of the invention) may comprise the steps of:
a) providing a set, collection or library of cells, derived from a Camelid, that express immunoglobulin sequences;
b) screening said set, collection or library of cells for (i) cells that express immunoglobulin sequences that can bind to and/or have affinity for a serum protein or at least one part, fragment, epitope or domain thereof; and (ii) cells that express heavy chain antibodies; in which substeps (i) and (ii) can be performed essentially as a single screening step or in any suitable order as two separate screening steps, so as to provide at least one cell that expresses heavy chain antibody that can bind to and/or has affinity for at least one domain or epitope of a serum protein;
c) determining the nucleotide sequence and/or the amino acid sequence of at least one heavy chain antibody, expressed by a cell provided in step b), that can bind to and/or has affinity for a serum protein or at least one part, fragment, epitope or domain thereof; and/or determining the nucleotide sequence and/or the amino acid sequence of a CDR sequence thereof and/or of a fragment thereof that comprises a CDR sequence;
d) preparing, using any suitable technique known per se, an amino acid sequence according of the invention that (i) essentially consist of a CDR sequence with an amino acid sequence that has been determined in step c); and/or (ii) comprises a fragment of an immunoglobulin with an amino acid sequence that has been determined in step c); and/or (iii) comprises a CDR sequence with an amino acid sequence that has been determined in step c), but that does not comprise an immunoglobulin fold and are also not capable of forming an immunoglobulin fold.

In this method, the collection or sample of cells is preferably a collection or sample of B-cells, and in particular a collection or sample of (B-)cells is obtained from a Camelid that has been suitably immunized with an antigen that comprises the desired domain or epitope(s) of a serum protein, such that an immune response against the desired domain or epitope(s) is raised.

The screening step b) may for example performed using a flow cytometry technique such as FACS.

In another specific method, nucleotide sequences are provided that encode amino acid sequences (such as CDR sequences) that can bind to a serum protein and that can be used in the present invention (i.e. as an amino acid sequence of the invention or as a starting point for providing an amino acid sequence of the invention). Such a method may comprise the steps of:
a) providing a set, collection or library of nucleotide sequences that encode amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold;
b) screening said set, collection or library for nucleotide sequences that encode amino acid sequences that can bind to and/or have affinity for a serum protein or at least one part, fragment, epitope or domain thereof; and
c) isolating the nucleotide sequence(s) that encode amino acid sequence(s) that can bind to and/or have affinity for said serum protein or said at least one part, fragment, epitope or domain thereof.

In step b) of such a method, said set, collection or library of nucleotide sequences is preferably screened for nucleotide sequences that encode amino acid sequences that can bind to and/or have affinity for a serum protein chosen from the group consisting of serum albumin, serum immunoglobulins such as IgG, thyroxine-binding protein, transferring or fibrinogen; and/or nucleotide sequences that encode for amino acid sequences that can bind to and/or have affinity for at least one part, fragment, epitope or domain of serum albumin, serum immunoglobulins such as IgG, thyroxine-binding protein, transferrin or fibrinogen.

In particular, in step b) of such a method, said set, collection or library of nucleotide sequences may be screened for nucleotide sequences that encode amino acid sequences that can bind to and/or have affinity for serum albumin or at least one part, fragment, epitope or domain thereof; and more in particular for nucleotide sequences that encode amino acid sequences that can bind to and/or have affinity for human serum albumin or at least one part, fragment, epitope or domain thereof. According to one specific, but non-limiting aspect, in step b) of such a method, said set, collection or library of nucleotide sequences may be screened for one or more nucleotide sequences that encode amino acid sequences that can bind to and/or have affinity for a part, fragment, epitope or domain of (human) serum albumin that is not involved in binding of (human) serum albumin to FcRn; and/or for nucleotide sequences that encode amino acid sequences that can bind to and/or have affinity for at least one part, fragment, epitope or domain of (human) serum albumin that does not form part of domain III of (human) serum albumin.

Said screening may be performed in any manner for protein screening known per se. For example, the amino acid sequences encoded by the set, collection or library of nucleotide sequences may be displayed on a phage, phagemid, ribosome or suitable micro-organism using techniques known to the skilled person. Reference is for example made to the review by Hoogenboom et al, Nat Biotechnol 23:1105, 2005 and the further prior art cited therein.

The set, collection or library of nucleotide sequences used in the above method preferably comprises a set, collection or library of nucleotide sequences that encode amino acid sequences that essentially consist of a CDR sequence flanked by two flanking amino acid sequences that have been derived from the immunoglobulin framework sequences; and/or a set, collection or library of nucleotide sequences that encode fragments of immunoglobulin sequences that comprise a CDR sequence flanked on both sides by framework sequences or fragments of framework sequences. In particular, the set, collection or library of nucleotide sequences may comprise a set, collection or library of nucleotide sequences that encode amino acid sequences that comprise or essentially consist of a CDR sequence flanked by two flanking amino acid sequences that have been derived from the framework sequences that, in the immunoglobulin variable domain from which said CDR sequence is derived, are adjacent to said CDR sequence. For example, the set, collection or library of nucleotide sequences may comprise or essentially consist of nucleotide sequences that encode CDR2 sequences flanked by two flanking amino acid sequences that have been derived from a framework 2 sequence and a framework 3 sequence, respectively; or of nucleotide sequences that encode CDR3 sequences flanked by two flanking amino acid sequences that have been derived from a framework 3 sequence and a framework 4 sequence, respectively.

For providing nucleotide sequences that encode amino acid sequences that contain two cysteine residues for forming a disulphide bridge (as further described herein), the above method may further comprise introducing (i.e. by adding, inserting or substituting one or more nucleotides) codons that encode one or two cysteine residues, such that each framework sequences in the amino acid sequence that is encoded by the nucleotide sequence thus obtained encodes contains at least one cysteine residue.

Alternatively, where the amino acid sequence encoded by the nucleotide sequence does not already comprise flanking amino acid sequences, the nucleotide sequence may be suitably linked to nucleotide sequences that encode such flanking sequences (preferably again with cysteine residues) may be added.

Also, one or more of the nucleotide sequences thus obtained may linked to each other and/or linked to one or more (and at least one) nucleotide sequences that encode a therapeutic moiety that comprises or essentially consists of an amino acid sequence (optionally via one or more nucleotide sequence that encode one or more linkers).

The above method may also comprise a step of suitably expressing the nucleotide sequence thus obtained, so as to provide an amino acid sequence of the invention or a polypeptide of the invention.

The set, collection or library of nucleotide sequences that is used in step a) of the above method (and that is subsequently screened in step b)) may be any suitable set, collection or library of nucleotide sequences that encode amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold. For example, it may be a set, collection or library of nucleotide sequences that encode amino acid sequences that has been obtained by method that comprises the use of one or more techniques for affinity maturation known per se.

However, according to one preferred aspect, such a set, collection or library may be obtained by a method that at least comprises the steps of:
a) providing a set, collection or library of nucleotide sequences that encode immunoglobulin sequences;
b) amplifying said nucleotide sequences using a combination of site-specific primers, such that the amplified fragments encode a set, library or collection of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold.

The set, collection or library of nucleotide sequences that encode immunoglobulin sequences that is used in step a) of the above method is may be any suitable set, collection or library of nucleotide sequences that encode immunoglobulin sequences, but may in particular be an immune set, collection or library, and more in particular an immune set, collection or library that has been obtained from mammal that has been suitably immunized with a serum protein (i.e. so as to raise an immune response against said serum protein). This set, collection or library may be generated in any manner known per se, such as by repertoire cloning (see for example WO 90/05144 or the review by Hoogenboom cited herein).

In one specific, but non-limiting aspect, said set, collection or library of nucleotide sequences that encode immunoglobulin sequences may be an immune set, collection or library of nucleotide sequences that encode heavy chain antibodies or VHH sequences, that have been obtained from a Camelid that has been suitably immunized with serum protein (i.e. so as to raise an immune response against said serum protein). For this, reference is for example made to the prior art cited herein.

The amplification step b) is preferably performed using (a combination of) site-specific primers that are specific for and/or capable of hybridizing to (i.e. under the conditions used for the amplification) nucleotide sequences that encode the framework sequences that flank said CDR sequence. For example, said (combination of) site-specific primers may be such that the amplified fragments encode a set, library or collection of amino acid sequences that (i) essentially consist of a CDR2 sequence; and/or (ii) comprise a fragment of an immunoglobulin that comprises a CDR2 sequences; and/or (iii) comprise a CDR2 sequence but that do not comprise an immunoglobulin fold and are also not capable of forming an immunoglobulin fold; in which case said site-specific primers may be specific for and/or capable of hybridizing to (i.e. under the conditions used for the amplification) nucleotide sequences that encode framework 2 sequences and framework 3 sequences, respectively.

Alternatively, said (combination of) site-specific primers may be such that the amplified fragments encode a set, library or collection of amino acid sequences that (i) essentially consist of a CDR3 sequence; and/or (ii) comprise a fragment of an immunoglobulin that comprises a CDR3 sequences; and/or (iii) comprise a CDR3 sequence but that do not comprise an immunoglobulin fold and are also not capable of forming an immunoglobulin fold; in which case said site-specific primers may be specific for and/or capable of hybridizing to (i.e. under the conditions used for the amplification) nucleotide sequences that encode framework 3 sequences and framework 4 sequences, respectively.

Some preferred, but non-limiting primers that can be used in the methods described herein are given in FIG. 1 and in SEQ ID NO's: 7 to 25. These primers (as well as similar primers that have at least 80%, such as at least 90%, for example at least 95% sequence identity with at least one of the primers of SEQ ID NO's: 7 to 25) form a further aspect of the invention.

Another aspect of the invention comprises the use of a suitable combination of at least two primers (i.e. at least one forward primer and at least one reverse primer) chosen from the primers of SEQ ID NO's: 7 to 25 (or from primers that have at least 80%, such as at least 90%, for example at least 95% sequence identity with at least one of the primers of SEQ ID NO's: 7 to 25) in generating (i.e. through amplification and optionally one or more of the further steps mentioned herein, such as affinity maturation) at least one amino acid sequence of the invention.

The amino acid sequences of the invention that may be generated using such primers (i.e. through amplification, optionally followed by one or more of the further steps mentioned herein, such as affinity maturation) form a further aspect of the invention. As the primers of SEQ ID NO's: 7 to 25 (and their variants) are suitable for amplifying CDR3 sequences, such an amino acid sequence is preferably a CDR3 sequence. Also, when the primers of SEQ ID NO's: 7 to 25 (or variants thereof) are used, the amino acid sequences obtained after amplification will usually contain flanking sequences on both end of the CDR3 sequence (see the examples below). However, this aspect of the invention also comprises the CDR3 sequences obtained with the primers of SEQ ID NO's: 7 to 25 (or variants thereof) without such flanking sequences, or with one or more other flanking sequences (as further described herein).

Such amino acid sequences (i.e. comprising CDR3 with or without flanking sequences) may further be as described herein, and are preferably in accordance with the preferred aspects described herein. For example, such amino acid sequences may be in a constrained or a non-constrained format; and/or may be such that they are capable of binding (as described herein) to a serum albumin, and in particular to human serum albumin, in a constrained format, in a non-constrained format, and preferably in both a constrained and non-constrained format.

In particular, such amino acid sequences (or a compound of the invention comprising at least one such amino acid sequence, as further described herein) are preferably such that they can bind to a serum albumin, and in particular to human serum albumin:
with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or
with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁴ s⁻¹, such as between 10⁻⁴ s⁻¹ and 10⁻⁶ s⁻¹;

Compounds of the invention that comprise one or more of the above amino acid sequences form a further specific aspect of the invention, and such compounds of the invention may be as further described herein (and are preferably in accordance with the preferred aspects described herein for compounds of the invention).

Other aspects of the invention comprise methods of the invention (which may be as further described herein), in which a suitable combination of at least two primers (i.e. at least one forward primer and at least one reverse primer) chosen from the primers of SEQ ID NO's: 7 to 25 (or from primers that have at least 80%, such as at least 90%, for example at least 95% sequence identity with at least one of the primers of SEQ ID NO's: 7 to 25) is used.

In yet another aspect, the invention relates to amino acid sequences that can be obtained (or have been obtained) by a method that comprises (the steps of) one of the methods described herein (and in particular one of the preferred methods described herein) followed by (at least one step of) affinity maturation (i.e. for improving the affinity for a serum protein, in particular a serum albumin, and more in particular human serum albumin). Such affinity maturation may be performed in any manner known per se for affinity maturation of proteins or polypeptides, and suitable methods and techniques will be clear to the skilled person based on the disclosure herein. Preferably, such affinity-matured amino acid sequences of the invention have an affinity for the relevant serum protein (such as a serum albumin, and more in particular human serum albumin) that is at least a factor 10 or more, such as a factor 100 or more, or even a factor 1000 or 10000 or more better than the affinity of the sequence used as the starting sequence for the affinity maturation step(s) (i.e. as obtained by one of the methods described herein).

Such affinity-matured amino acid sequences of the invention may further be as described herein, and are preferably in accordance with the preferred aspects described herein. For example, such amino acid sequences may be in a constrained or a non-constrained format; and/or may be such that they are capable of binding (as described herein) to a serum albumin, and in particular to human serum albumin, in a constrained format, in a non-constrained format, and preferably in both a constrained and non-constrained format.

In particular, such amino acid sequences (or a compound of the invention comprising at least one such amino acid sequence, as further described herein) are preferably such that they can bind to a serum albumin, and in particular to human serum albumin:
with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻;
   and/or
with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁴ s⁻¹, such as between 10⁻⁴ s⁻¹ and 10⁻⁶ s⁻¹;

Compounds of the invention that comprise one or more of the above amino acid sequences form a further specific aspect of the invention, and such compounds of the invention may be as further described herein (and are preferably in accordance with the preferred aspects described herein for compounds of the invention).

In another aspect, the invention relates to an amino acid sequence that can bind to a serum protein and that comprises at least one (and preferably only one) disulfide bridge.

Such an amino acid sequence preferably has a length of less than 90 amino acid residues, preferably less than 50 amino acid residues, such as about 40, 30 or 20 amino acid residues.

For example, according to a preferred but non-limiting aspect, such an amino acid sequence comprises or essentially consists of a peptide sequence that can bind to a serum protein flanked by two flanking amino acid sequences, in which each flanking amino acid sequence contains a cysteine residue that forms part of the disulfide bridge. In such an amino acid sequence, the peptide sequence may have a length between 3 and 30 amino acid residues, preferably between 5 and 25 amino acid residues, and the two flanking amino acid sequences may each have a length of between 1 and 30 amino acid residues, preferably between 2 and 20 amino acid residues, such as about 5, 10 or 15 amino acid residues.

Again, in such amino acid sequences, the two flanking amino acid sequences are preferably derived from immunoglobulin framework sequences and/or are preferably fragments of immunoglobulin framework sequences, in which the cysteine residue in each flanking amino acid sequence that forms part of the disulphide bridge is either a cysteine residue that naturally occurs in said immunoglobulin framework sequences (or in said fragment thereof) and/or is a cysteine residue that has been introduced into said in immunoglobulin framework sequence (or in said fragment thereof).

The peptide sequence present in these amino acid sequences may be a synthetic peptide sequence, a peptide sequence that has been generated using an affinity maturation technique, or may essentially consists of a CDR sequence (i.e. as further described herein). Again, such a CDR sequence may have been derived from an V_{H}-, V_{L}- or V_{HH}-sequence that can bind to a serum protein, and in particular from a (single) domain antibody, a dAb, or a Nanobody® or a fragment thereof.

Again, a CDR2 sequence (in which case one of the two flanking amino acid sequences is preferably derived from a framework 2 sequence and/or a fragment of a framework 2 sequence, and the other flanking amino acid sequence is preferably derived from a framework 3 sequence and/or is a fragment of a framework 3 sequence) and a CDR3 sequence (in which case one of the two flanking amino acid sequences is preferably derived from a framework 3 sequence and/or a fragment of a framework 3 sequence, and the other flanking amino acid sequence is preferably derived from a framework 4 sequence and/or is a fragment of a framework 4 sequence) are particularly preferred.

Again, such an amino acid sequence preferably can bind to a serum protein in such a way that the half-life of the serum protein molecule is not (significantly) reduced. Also, again, such an amino acid sequence preferably can bind to a serum protein from the group consisting of serum albumin, serum immunoglobulins, thyroxine-binding protein, transferrin, fibrinogen or fragments thereof; and in particular to a serum albumin or a fragment thereof; and more in particular to human serum albumin or a fragment thereof. When the amino acid sequence can bind to (human) serum albumin, it is preferably capable of binding to amino acid residues on (human) serum albumin that are not involved in binding of serum albumin to FcRn; and/or to amino acid residues on (human) serum albumin that do not form part of domain III of serum albumin.

Also, the at least one therapeutic moiety preferably comprises or essentially consists of an amino acid sequence, and in particular of an immunoglobulin sequence or an antigen-binding fragment thereof (for example, an antibody or an antigen-binding fragment thereof), such as an immunoglobulin variable domain or an antigen-binding fragment thereof (for example, a V_{H}-domain, a V_{L}-domain, a V_{HH}-domain or an antigen-binding fragment thereof); or a protein or polypeptide comprising the same (for example, an scFv construct); and more in particular a (single) domain antibody, a “dAb”, or a Nanobody®.

The invention also relates to compounds or constructs that comprise at least one such amino acid sequence and at least one therapeutic moiety. Again, the at least one amino acid sequence may either be directly linked to the at least one therapeutic moiety or may be linked to the at least one therapeutic moiety via one or more suitable linkers or spacers. When the at least one therapeutic moiety preferably comprises or essentially consists of an amino acid sequence, the linkers or spacers also preferably comprise or essentially consist of amino acid sequences, such that the resulting compound or construct comprises or essentially consist of a (fusion) protein or (fusion) polypeptide (i.e. with one disulphide bridge in each amino acid sequence of the invention).

The invention also relates to a nucleotide sequence or nucleic acid that encodes an amino acid sequence with the same primary amino acid sequence as an amino acid sequence of the invention (i.e. with the two cysteine residues capable of forming a disulphide bridge, but without the disulphide bridge itself) or that encodes an amino acid sequence with the same primary amino acid sequence as a polypeptide of the invention (i.e. with the two cysteine residues capable of forming a disulphide bridge, but without the disulphide bridge itself).

The invention also relates to a host or host cell that contains such a nucleotide sequence or nucleic acid and/or that expresses (or is capable of expressing) an amino acid sequence with the same primary amino acid sequence as an amino acid sequence of the invention (i.e. with the two cysteine residues capable of forming a disulphide bridge, but without the disulphide bridge itself) or an amino acid sequence with the same primary amino acid sequence as a polypeptide of the invention (i.e. with the two cysteine residues capable of forming a disulphide bridge, but without the disulphide bridge itself).

The invention also relates to a method for preparing a desired amino acid sequence compound or construct of the invention (i.e. comprising at least one disulphide bridge as described herein), which comprises at least the steps of:
a) providing an amino acid sequence with the same primary amino acid sequence as the desired amino acid sequence of the invention (i.e. with the two cysteine residues capable of forming a disulphide bridge, but without the disulphide bridge itself) or with the same primary amino acid sequence as the desired polypeptide of the invention (i.e. with the two cysteine residues capable of forming a disulphide bridge, but without the disulphide bridge itself); and
b) forming a disulphide bridge in said amino acid sequence so as to provide the desired amino acid sequence of the invention or the desired compound or construct of the invention, respectively.

In particular, when the desired compound or construct is a polypeptide of the invention, such a method may at least comprise the steps of:
a) expressing a nucleotide sequence or nucleic acid that encodes an amino acid sequence with the same primary amino acid sequence as the desired amino acid sequence of the invention (i.e. with the two cysteine residues capable of forming a disulphide bridge, but without the disulphide bridge itself) or that encodes an amino acid sequence with the same primary amino acid sequence as the desired polypeptide of the invention (i.e. with the two cysteine residues capable of forming a disulphide bridge, but without the disulphide bridge itself), so as to provide an amino acid sequence with the same primary amino acid sequence as the desired amino acid sequence of the invention to 138 or an amino acid sequence with the same primary amino acid sequence as the desired polypeptide of the invention, respectively;
   and optionally further comprises:
b) isolating the amino acid sequence obtained in step b);
   and:
c) forming a disulphide bridge in the amino acid sequence obtained in step a) or, when step b) is performed, in the amino acid sequence obtained in step b), respectively, so as to provide the desired amino acid sequence of the invention or the desired compound or construct of the invention, respectively.

The invention also relates to an amino acid sequence, compound or construct that is obtained via any of the above methods.

The invention further relates to a pharmaceutical composition that comprises at least one amino acid sequence, at least one compound or construct, or at least one nucleotide sequence as described herein; and optionally at least one pharmaceutically acceptable carrier, diluent or excipient.

The invention also encompasses some other methods for preparing the constructs and compounds described herein, which generally comprise the step of linking at least one amino acid sequence of the invention to at least one therapeutic moiety, optionally via one or more suitable linkers or spacers. This may be performed in any suitable manner known per se, for example depending on the linker(s) used (if any), and may for example comprise techniques for chemical linking known per se in the art, for example by formation of one or more covalent bonds. The one or more amino acid sequences of the invention and the one or more therapeutic moieties may be as further described herein. Again, the one or more amino acid sequences of the invention preferably comprise a disulphide bridge as described herein.

The invention also relates to compound or construct that is obtained via any of the above methods; and also to a pharmaceutical composition that comprises at least one such compound or construct and optionally at least one pharmaceutically acceptable carrier, diluent or excipient.

### DETAILED DESCRIPTION OF THE INVENTION

In the present description, examples and claims:
a) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, N.Y. (2005), as well as to the general background art cited herein;
b) Unless indicated otherwise, the term “immunoglobulin sequence”—whether used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation;
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2(1): 49-57; Irving et al., J. Immunol. Methods, 2001, 248(1-2), 31-45; Schmitz et al., Placenta, 2000, 21 Suppl. A, S106-12, Gonzales et al., Tumour Biol., 2005, 26(1), 31-43, which describe techniques for protein engineering, such as affinity maturation and other techniques for improving the specificity and other desired properties of proteins such as immunoglobulins.
d) Amino acid residues will be indicated according to the standard three-letter or one-letter amino acid code, as mentioned in Table A-2;

TABLE A-2
one-letter and three-letter amino acid code

| | | | |
|---|---|---|---|
| Nonpolar, | Alanine | Ala | A |
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residue is greatly dependant upon even small shifts in pH, but a His residue can generally be considered essentially uncharged at a pH of about 6.5.

e) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).
   Alternatively, the degree of sequence identity between two or more nucleotide sequences may be calculated using a known computer algorithm for sequence alignment such as NCBI Blast v2.0, using standard settings.
   Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.
   Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
f) For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings.
   Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.
   Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.
   Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.
   Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Natl. Acad Sci. USA 81: 140-144, 1984; Kyte & Doolittle; J Molec. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a VHH domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first-mentioned nucleotide sequence or amino acid sequence, respectively, but more usually this generally means that the first-mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first-mentioned sequence has actually been generated or obtained (which may for example be by any suitable method described herein). By means of a non-limiting example, when a Nanobody of the invention is said to comprise a CDR sequence, this may mean that said CDR sequence has been incorporated into the Nanobody of the invention, but more usually this generally means that the Nanobody of the invention contains within its sequence a stretch of amino acid residues with the same amino acid sequence as said CDR sequence, irrespective of how said Nanobody of the invention has been generated or obtained. It should also be noted that when the latter amino acid sequence has a specific biological or structural function, it preferably has essentially the same, a similar or an equivalent biological or structural function in the first-mentioned amino acid sequence (in other words, the first-mentioned amino acid sequence is preferably such that the latter sequence is capable of performing essentially the same, a similar or an equivalent biological or structural function). For example, when a Nanobody of the invention is said to comprise a CDR sequence or framework sequence, respectively, the CDR sequence and framework are preferably capable, in said Nanobody, of functioning as a CDR sequence or framework sequence, respectively. Also, when a nucleotide sequence is said to comprise another nucleotide sequence, the first-mentioned nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the first-mentioned, larger nucleotide sequence).
j) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 1 0-fold, more in particular at least 100-fold, and up to 1 000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
k) The term “domain” as used herein generally refers to a globular region of an amino acid sequence (such as an antibody chain, and in particular to a globular region of a heavy chain antibody), or to a polypeptide that essentially consists of such a globular region. Usually, such a domain will comprise peptide loops (for example 3 or 4 peptide loops) stabilized, for example, as a sheet or by disulfide bonds. The term “binding domain” refers to such a domain that is directed against an antigenic determinant (as defined herein);
l) The term “antigenic determinant” refers to the epitope on the antigen recognized by the antigen-binding molecule (such as a Nanobody or a polypeptide of the invention) and more in particular by the antigen-binding site of said molecule. The terms “antigenic determinant” and “epitope” may also be used interchangeably herein.
m) An amino acid sequence (such as a Nanobody, an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
n) The term “specificity” refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the amino acid sequences, Nanobodies and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired serum protein with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.
   The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e.g., of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].
   The affinity denotes the strength or stability of a molecular interaction. The affinity is commonly given as by the K_{D}, or dissociation constant, which has units of mol/liter (or M). The affinity can also be expressed as an association constant, K_{A}, which equals 1/K_{D} and has units of (mol/liter)⁻¹ (or M⁻¹). In the present specification, the stability of the interaction between two molecules (such as an amino acid sequence, Nanobody or polypeptide of the invention and its intended target) will mainly be expressed in terms of the K_{D} value of their interaction; it being clear to the skilled person that in view of the relation K_{A}=1/K_{D}, specifying the strength of molecular interaction by its K_{D} value can also be used to calculate the corresponding K_{A} value. The K_{D}-value characterizes the strength of a molecular interaction also in a thermodynamic sense as it is related to the free energy (DG) of binding by the well known relation DG=RT·ln(K_{D}) (equivalently DG=−RT·ln(K_{A})), where R equals the gas constant, T equals the absolute temperature and In denotes the natural logarithm.
   The K_{D} for biological interactions which are considered meaningful (e.g. specific) are typically in the range of 10⁻¹⁰M (0.1 nM) to 10⁻⁵M (10000 nM). The stronger an interaction is, the lower is its K_{D}.
   The K_{D} can also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate may vary between 10⁻⁶ s⁻¹ (near irreversible complex with a t_{1/2} of multiple days) to 1 s⁻¹ (t_{1/2}=0.69 s).
   The affinity of a molecular interaction between two molecules can be measured via different techniques known per se, such as the well the known surface plasmon resonance (SPR) biosensor technique (see for example Ober et al., Intern. Immunology, 13, 1551-1559, 2001) where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIACORE instruments.
   It will also be clear to the skilled person that the measured K_{D} may correspond to the apparent K_{D} if the measuring process somehow influences the intrinsic binding affinity of the implied molecules for example by artifacts related to the coating on the biosensor of one molecule. Also, an apparent K_{D} may be measured if one molecule contains more than one recognition sites for the other molecule. In such situation the measured affinity may be affected by the avidity of the interaction by the two molecules.
   Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artifacts relating to adsorption of one of the molecules on a support such as plastic.
   However, the accurate measurement of K_{D} may be quite labor-intensive and as consequence, often apparent K_{D} values are determined to assess the binding strength of two molecules. It should be noted that as long all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance. Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labeled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of A is varied for a given concentration or amount of B. As a result an IC₅₀ value is obtained corresponding to the concentration of A at which the signal measured for C in absence of A is halved. Provided K_{D ref}, the K_{D} of the reference molecule, is known, as well as the total concentration c_{ref} of the reference molecule, the apparent K_{D} for the interaction A-B can be obtained from following formula: K_{D}=IC₅₀/(1+c_{ref}/K_{D ref}). Note that if c_{ref}<<K_{D ref}, K_{D}≈IC₅₀. Provided the measurement of the IC₅₀ is performed in a consistent way (e.g. keeping c_{ref} fixed) for the binders that are compared, the strength or stability of a molecular interaction can be assessed by the IC₅₀ and this measurement is judged as equivalent to K_{D} or to apparent K_{D} throughout this text.
o) The half-life of an amino acid sequence, compound or polypeptide of the invention can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally involve the steps of suitably administering to a warm-blooded animal (i.e. to a human or to another suitable mammal, such as a mouse, rabbit, rat, pig, dog or a primate, for example monkeys from the genus Macaca (such as, and in particular, cynomolgus monkeys (Macaca fascicularis) and/or rhesus monkeys (Macaca mulatta)) and baboon (Papio ursinus)) a suitable dose of the amino acid sequence, compound or polypeptide of the invention; collecting blood samples or other samples from said animal; determining the level or concentration of the amino acid sequence, compound or polypeptide of the invention in said blood sample; and calculating, from (a plot of) the data thus obtained, the time until the level or concentration of the amino acid sequence, compound or polypeptide of the invention has been reduced by 50% compared to the initial level upon dosing. Reference is for example made to the Experimental Part below, as well as Dennis et al., J. Biol. Chem. 277:35035-42 (2002).to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982).
   As will also be clear to the skilled person (see for example pages 6 and 7 of WO 04/003019 and in the further references cited therein), the half-life can be expressed using parameters such as the t½-alpha, t½-beta and the area under the curve (AUC).

In the present specification, an “increase in half-life” refers to an increase in any one of these parameters, such as any two of these parameters, or essentially all three these parameters. As used herein “increase in half-life” or “increased half-life” in particular refers to an increase in the t½-beta, either with or without an increase in the t½-alpha and/or the AUC or both.
p) Any Figures, Sequence Listing and the Experimental Part/Examples are only given to further illustrate the invention and should not be interpreted or construed as limiting the scope of the invention and/or of the appended claims in any way, unless explicitly indicated otherwise herein.

For a general description of heavy chain antibodies and the variable domains thereof, reference is inter alia made to the prior art cited herein, to the review article by Muyldermans in Reviews in Molecular Biotechnology 74 (2001), 277-302; as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. Reference is also made to the further prior art mentioned in these applications, and in particular to the list of references mentioned on pages 41-43 of the International application WO 06/040153, which list and references are incorporated herein by reference.

In accordance with the terminology used in the art (see the above references), the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”). As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins.

The amino acid sequences of the invention are preferably of mammalian origin, or are derived from (as defined herein) an amino acid sequence of mammalian origin. For example, the amino acid sequences may be derived from a species of mammal that produces heavy-chain antibodies, such as Camelids or transgenic animals carrying such heavy chain antibody locus (see for example WO 02/085945, WO 04/049794, WO 06/008548 and Janssens et al., Proc. Natl. Acad. Sci. USA. 2006 Oct. 10; 103(41):15130-5) can be used. Alternatively, the amino acid sequences of the invention may be derived from single variable domains as may occur in certain species of sharks (for example, the so-called “IgNAR domains”, see for example WO 05/18629).

The amino acid sequences of the invention preferably comprise or essentially consist of a CDR (‘complementary determining region’) sequence (also referred to herein as “CDR sequences”); such CDR sequences preferably can bind to serum proteins and can be derived from immunoglobulin variable domain sequences that have been raised and/or directed against, a serum protein, in particular against a serum albumin, and more in particular against a human serum albumin (or against a part, domain or fragment thereof). According to a preferred, but non-limiting aspect, the CDR sequences are derived from CDR2 sequences or CDR3 sequences from immunoglobulin variable domains. Such immunoglobulin variable domains can be, for example, human variable domains, (single domain antibodies), dAb's, Nanobodies® or functional fragments thereof.

The invention also provides methods for identifying and generating such peptides and for preparing compounds, proteins, polypeptides, fusion proteins and constructs, comprising at least one such peptide.

According to a preferred but non-limiting aspect, the amino acid sequences of the invention are derived from an immunoglobulin heavy chain variable domain. Preferred examples of such heavy chain variable domains are V_{HH} domains from Camelidae.

According to another specific but non-limiting aspect, the amino acid sequences of the invention comprises or essentially consists of a CDR sequence, which has a length in the range of 3 to 40 amino acids, preferably in the range of 5 to 30 amino acids, more preferably in the range of 6 to 25 amino acids; the length of an amino acid sequence of the invention may be (but is not limited to) for example 8 amino acids, 10 amino acids, 12 amino acids, 14 amino acids, 16 amino acids, 18 amino acids, 20 amino acids, 22 amino acids or 24 amino acids.

The amino acid sequences of the invention (as well as compounds comprising the same, as defined herein) are preferably such that they bind to or otherwise associate with human serum albumin in such a way that, when the amino acid sequence (or compound) is bound to or otherwise associated with a human serum albumin in man, it exhibits a serum half-life of at least about 50% (such as about 50% to 70%), preferably at least 60% (such as about 60% to 80%), or preferably at least 70% (such as about 70% to 90%), more preferably at least 80% (such as about 80% to 90%), or preferably at least about 90% of the natural half-life of the human serum albumin in man.

In one non-limiting aspect, the amino acid sequences of the invention are preferably cross-reactive with serum albumin from at least one other species of mammal, for example from mouse, rabbit, rat, or a primate. In particular, the amino acid sequences of the invention may be cross-reactive with serum albumin from a primate chosen from the group consisting of monkeys from the genus Macaca (such as, and in particular, cynomolgus monkeys (Macaca fascicularis) and/or rhesus monkeys (Macaca mulatta) and baboon (Papio ursinus). Also, when an amino acid sequence of the invention is cross-reactive with serum albumin from such a species of primate, it is preferably such that, when it is bound to or associated with a serum albumin molecule in said primate, it exhibits a serum half-life of at least about 50% (such as about 50% to 70%), preferably at least about 60% (such as about 60% to 80%), or preferably at least about 70% (such as about 70% to 90%), more preferably at least about 80% (such as about 80% to 90%), or preferably at least about 90% of the natural half-life of said serum albumin in said primate.

Generally, the compounds or polypeptides of the invention that comprise at least one amino acid sequence of the invention and at least one therapeutic moiety preferably have a half-life that is at least 1.5 times, preferably at least 2 times, such as at least 5 times, for example at least 10 times or more than 20 times, greater than the half-life of the therapeutic moiety per se. For example, the compounds or polypeptides of the invention may have a half-life that is increased with more than 1 hours, preferably more than 2 hours, more preferably more than 6 hours, such as more than 12 hours, or even more than 24, 48 or 72 hours, compared to the therapeutic moiety per se.

In a preferred, but non-limiting aspect of the invention, such compounds or polypeptides of the invention have a serum half-life that is increased with more than 1 hours, preferably more than 2 hours, more preferably more than 6 hours, such as more than 12 hours, or even more than 24, 48 or 72 hours, compared to the therapeutic moiety per se.

In another preferred, but non-limiting aspect, the amino acid sequences of the invention are preferably such that they bind to or otherwise associate with human serum albumin in such a way that, when the amino acid sequences are bound to or otherwise associated with a human serum albumin, the amino acid sequences exhibit a serum half-life in human of at least about 9 days (such as about 9 to 14 days), preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

The amino acid sequences of the invention also preferably bind to human serum albumin with a dissociation constant (K_{D}) and/or with a binding affinity (K_{A}) that is as defined herein.

The invention also relates to compounds i.e. a compound, protein, polypeptide or other construct that comprises at least one amino acid sequence of the invention and at least one therapeutic moiety, such as at least one moiety chosen from the group consisting of small molecules, polynucleotides, polypeptides or peptides that have a half-life in human that is at least 80%, more preferably at least 90%, such as 95% or more, or essentially the same as the half-life in human of the amino acid sequence of the invention.

In this description, the term “sequence” in particular refers to an amino acid sequence and/or to a nucleotide/nucleic acid sequence, depending on what the context requires. When a sequence is in the form of a nucleic acid sequence, the corresponding amino acid sequence may be prepared by suitably expressing the amino acid sequence encoded by said nucleic acid sequence.

With “comprise or essentially consist of” is in particular meant in this description that the amino acid sequences of the invention can contain one or more additional amino acid sequences, which have a length of, for example but not limited to, 1 to 10 amino acid residues, such as 1, 2, 3, 4, 5, 6, 7, 8, 9, or 10, (which are not derived from CDR sequences) at either or both ends of the CDR sequence. These additional amino acid sequences can be derived from framework sequences, for example the framework sequences that are adjacent to the CDR sequence in a full size immunoglobulin sequence; for example if the amino acid sequence of the invention comprises or essentially consists of a CDR sequence, which is derived from a CDR3 sequence, the additional amino acid sequences can be derived from framework 3 and or framework 4 regions. According to a non-limiting preferred embodiment, the additional amino acid sequences can comprise at least two cysteine residues (i.e. on either side of the serum protein binding sequence) which may or may not be linked via a disulfide bridge. For example, when the additional amino acid sequences are derived from framework sequences, cysteine residues may be introduced synthetically; alternatively, other additional amino acid residues may be chosen, which contain such (naturally occurring) cysteine residues.

When in this description, a sequence (amino acid or nucleic acid) is in particular said to be “derived from” another sequence (amino acid or nucleic acid), the desired sequence (amino acid or nucleic acid) can be obtained by generating and/or isolating the relevant sequence and subsequently isolating the relevant part(s) thereof, or by directly generating and/or isolating the relevant part(s) of said other sequence; both in a manner known per se.

Alternatively, the sequence (amino acid or nucleic acid) of said other sequence can be determined, after which the desired sequence can be prepared in a manner known per se, using the determined sequence as a starting point. For example, a desired amino acid sequence may be prepared by peptide synthesis or by suitably expressing a nucleic acid encoding said amino acid sequence. A desired nucleotide sequence may be prepared by techniques of nucleic acid synthesis known per se.

Parts, fragments, variants, analogs, etc. of a desired sequence (amino acid or nucleic acid) may be prepared using techniques known per se, such as digesting with restriction enzymes, suitably linking one or more sequences, site-directed mutagenesis, PCR using one or more primers that introduce the desired mutation(s), de novo synthesis (amino acid or nucleic acid), and/or by any suitable combination of such techniques; or in any other suitable manner known per se.

When in this specification, a sequence (amino acid or nucleic acid) is said to be “derived from” a (species of) mammal, said sequence is “derived from” (as defined herein) from a sequence (amino acid or nucleic acid) that occurs naturally in said mammal.

Thus, for example, when in this description, reference is made to a sequence (amino acid or nucleic acid) that is “a CDR sequence derived from an immunoglobulin variable domain sequence”), said sequence (amino acid or nucleic acid) can in particular be obtained by isolating said other sequence (amino acid or nucleic acid) of an immunoglobulin variable domain CDR sequence or suitable parts, fragments, analogs, variants thereof;

Alternatively, its sequence (amino acid or nucleic acid) can be obtained by determining the sequence (amino acid or nucleic acid) of an immunoglobulin variable domain CDR sequence or suitable parts, fragments, analogs, variants thereof and subsequently using this sequence as a starting point for designing synthetic or semi-synthetic amino acid sequences of the invention, which may for example be parts, fragments, analogs, variants, etc. of the naturally occurring CDR sequence. In case the CDR sequence is an amino acid sequence, it can be obtained by any suitable peptide synthesis technique, known to the skilled person; alternatively, in case the CDR sequence is a nucleic acid sequence, it can be prepared by any suitable nucleic acid synthesis technique, known to the skilled person, and subsequently be expressed.

When in this description, reference is made to “immunoglobulin sequences raised against a serum protein” or “immunoglobulin sequences directed against a serum protein”, these immunoglobulin sequences were naturally produced by the human or animal body through activation of the immune system upon suitable introduction of the (preferably heterologous) serum protein into the blood circulation of the human or animal (i.e. so as to raise an immune response against the serum protein e.g. by means of suitable immunization with the serum protein).

When in this description, reference is made to “binding”, such binding is preferably specific binding, as normally understood by the skilled person. In particular, when an amino acid sequence as described herein “binds to a serum protein”, it is preferably such that it binds to said serum protein:
with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷ M⁻¹s⁻¹, such as between 10⁵ M⁻¹s⁻¹ and 10⁷ M⁻¹s⁻¹;
   and/or
with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁴ s⁻¹, such as between 10⁴ s⁻¹ and 10⁻⁶ s⁻¹.

Preferably, a monovalent amino acid sequence of the invention (or a polypeptide that contains only one amino acid sequence of the invention) is preferably such that it will bind to the serum protein with an affinity less than 500 nM, preferably less than 200 nM, more preferably less than 10 nM, such as less than 500 μM.

In another aspect, the invention provides amino acid sequences that can be used as small peptides or peptide moieties for linking or fusing to a therapeutic compound in order to increase the half-life thereof, and constructs and fusion proteins comprising such peptides or peptide moieties, that can bind to a serum protein in such a way that, when the amino acid sequence, construct, or fusion protein of the invention is bound to a serum protein molecule, the half-life of the serum protein molecule is not (significantly) reduced (i.e. compared to the half-life of the serum protein molecule when the amino acid sequence, construct, or fusion protein is not bound thereto). In this aspect of the invention, by “not significantly reduced” is meant that the half-life of the serum protein molecule (as measured using a suitable technique known per se) is not reduced by more than 50%, preferably not reduced by more than 30%, even more preferably not reduced by more than 10%, such as not reduced by more than 5%, or essentially not reduced at all.

The amino acid sequences of the invention preferably comprise or essentially consist of CDR sequences derived from immunoglobulin variable domains that have been raised and/or directed against a serum protein, in particular against serum albumin, and more in particular against human serum albumin (or against a part, domain or fragment thereof).

Said amino acid sequences may for example comprise or essentially consist of CDR sequences, such as for example CDR1, CDR2 or CDR3, derived from immunoglobulin variable domains.

Preferably, the amino acid sequences of the invention comprise or essentially consist of a CDR3 sequence. According to another specific but non-limiting aspect, the amino acid sequences of the invention may essentially be as described in WO 03/050531, referred to above and incorporated herein by reference.

The immunoglobulin variable domains from which the amino acid sequence may be derived can for example be (but are not limited to) immunoglobulin heavy or light chain variable domains, (single) domain antibodies, ‘dAbs’, or Nanobodies.

According to a particularly preferred embodiment, the amino acid sequences of the present invention comprise or essentially consist of CDR sequences of Nanobodies® that have been raised and/or directed against a serum protein, in particular against serum albumin, and more in particular against human serum albumin (or against a part, domain or fragment thereof); more preferably the amino acid sequences of the present invention comprise or essentially consist of CDR3 sequences of Nanobodies® that have been raised and/or directed against a serum protein, in particular against serum albumin, and more in particular against human serum albumin (or against a part, domain or fragment thereof). Again, according to this specific aspect of the invention, the amino acid sequences of the invention may essentially be as described in WO 03/050531. For a further description and definition of Nanobodies®, as well as of some of the further terms used in the present description, reference is also made to the applications by Ablynx N.V. mentioned herein as well as the further prior art cited therein.

In other aspects, the invention also relates to methods for generating or producing the amino acid sequences of the invention (or compounds comprising the same).

For example, when the amino acid sequence of the invention is a CDR amino acid sequence (or a suitable part, analog, fragment, variant thereof), said method may comprise the steps of:
a) providing a set, collection or library of CDR sequences;
b) screening said set, collection or library of CDR sequences for sequences that can bind to and/or have affinity for at least one domain or epitope of a serum protein;
c) isolating the CDR sequence(s) that can bind to and/or have affinity for at least one domain or epitope of a serum protein.

This method may be performed in any manner known per se, using techniques known to the person skilled in the art and/or as further described herein. For example, methods for providing libraries of CDR sequences, for screening such libraries for sequences that have affinity for a desired target, and for isolating CDR sequences that bind to a desired antigen are described in WO 03/050531 (Ablynx N.V. and Algonomics N.V.).

In the above method, the set, collection or library of CDR sequences may for example be displayed on a phage, phagemid, ribosome or suitable micro-organism (such as yeast), such as to facilitate screening. Suitable methods, techniques and host organisms for displaying and screening (a set, collection or library of) CDR sequences will be clear to the person skilled in the art, for example on the basis of the further disclosure and prior art cited herein

Based on the sequence obtained in c), parts, analogs, fragments, variants of said sequence can be prepared in a manner known per se, for example by site-specific mutagenesis (using mismatched primers), by de novo nucleic acid synthesis, or de novo peptide synthesis.

Alternatively, CDR amino acid sequences or suitable parts, analogs, fragments, variants thereof that bind to serum proteins can be generated by a method provided by the invention, at least comprising the steps of:
a) providing a set, collection or library of immunoglobulin sequences;
b) screening said set, collection or library of immunoglobulin sequences for sequences that can bind to and/or have affinity for at least one domain or epitope of a serum protein;
c) optionally isolating the immunoglobulin sequence(s) that can bind to and/or have affinity for at least one domain or epitope of a serum protein;
d) preparing CDR sequences derived from the immunoglobulin sequences obtained in c) using techniques that are known by the skilled person.

This method may again be performed in any manner known per se, using techniques known to the person skilled in the art and/or as further described herein. Step d) may for example be performed by using suitable site specific primers, such as (but not limited to) a primer combination consisting of a FW3 (‘framework 3’)—specific and a FW4 (‘framework 4’)—specific primer and subsequently expressing the obtained (amplified) nucleic acid sequence.

Based on the sequence obtained in c) or d), parts, analogs, fragments, variants can be prepared in a manner known per se, for example by site-specific mutagenesis (using mismatched primers), by de novo nucleic acid synthesis, or de novo peptide synthesis.

In this method, the set, collection or library of immunoglobulin sequences may be a naïve set, collection or library of immunoglobulin sequences; a synthetic or semi-synthetic set, collection or library of immunoglobulin sequences; and/or a set, collection or library of immunoglobulin sequences that have been subjected to affinity maturation.

Also, in such a method, the set, collection or library of immunoglobulin sequences may be a set, collection or library of heavy chain variable domains or of light chain variable domains. For example, the set, collection or library of immunoglobulin sequences may be a set, collection or library of domain antibodies or single domain antibodies, or is a set, collection or library of immunoglobulin sequences that are capable of functioning as a domain antibody or single domain antibody.

In a preferred aspect of this method, the set, collection or library of immunoglobulin sequences may be an immune set, collection or library of immunoglobulin sequences, for example derived from a mammal that has been suitably immunized with an antigen that comprises the desired extracellular part, region, domain, loop or other extracellular epitope(s), such that an immune response against the desired extracellular part, region, domain, loop or other extracellular epitope(s) is raised. In one specific, but non-limiting aspect, the immune set, collection or library of immunoglobulin sequences may be derived from a Camelid.

The immune set, collection or library of immunoglobulin sequences may for example be a set, collection or library of heavy chain variable domains or of light chain variable domains. In one specific aspect, the set, collection or library of immunoglobulin sequences is a set, collection or library of VHH sequences.

In the above method, the set, collection or library of immunoglobulin sequences may be displayed on a phage, phagemid, ribosome or suitable micro-organism (such as yeast), such as to facilitate screening. Suitable methods, techniques and host organisms for displaying and screening (a set, collection or library of) immunoglobulin sequences will be clear to the person skilled in the art, for example on the basis of the further disclosure herein. Reference is also made to the review by Hoogenboom in Nature Biotechnology, 23, 9, 1105-1116 (2005) and the further prior art cited therein.

In another aspect, a method for generating CDR amino acid sequences or suitable parts, analogs, fragments, variants thereof that bind to serum proteins, at least comprises the steps of:
a) providing a collection or sample of cells expressing immunoglobulin sequences
b) screening said collection or sample of cells for cells that express an immunoglobulin sequence that can bind to and/or has affinity for at least one domain or epitope of a serum protein;
c) either (i) isolating from said cell the desired CDR sequence (optionally after first isolating from said cell said immunoglobulin sequence); or (ii) isolating from said cell a nucleic acid sequence that encodes the desired CDR sequence from said immunoglobulin sequence; followed by expressing the desired CDR sequence from said immunoglobulin sequence.

In the method according to this aspect, the collection or sample of cells may for example be a collection or sample of B-cells. Also, in this method, the sample of cells may be derived from a mammal that has been suitably immunized with an antigen that comprises the desired extracellular part, region, domain, loop or other extracellular epitope(s), such that an immune response against the desired extracellular part, region, domain, loop or other extracellular epitope(s) is raised. For example, the collection or sample of cells may be derived from a suitably immunized Camelid.

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820.

As mentioned above, in a preferred but non-limiting aspect, the amino acid sequences of the invention are preferably derived from a heavy chain antibody, and more preferably comprise or essentially consist of a CDR sequence (such as a CDR3 sequence) derived from a heavy chain antibody (or a part, fragment, analog or variant thereof). In this aspect, a preferred method for isolating CDR sequences from heavy chain antibodies at least comprises the steps of:
a) providing a collection or sample of cells expressing immunoglobulin sequences;
b) screening said collection or sample of cells for (i) cells that express an immunoglobulin sequence that can bind to and/or have affinity for at least one domain or epitope of a serum protein; and (ii) cells that express heavy chain antibodies, in which substeps (i) and (ii) can be performed essentially as a single screening step or in any suitable order as two separate screening steps, so as to provide at least one cell that expresses heavy chain antibody that can bind to and/or has affinity for at least one domain or epitope of a serum protein;
c) either (i) isolating from said cell the desired CDR sequence (optionally after first isolating from said cell said immunoglobulin sequence); or (ii) isolating from said cell a nucleic acid sequence that encodes the desired CDR sequence, followed by expressing said CDR sequence.

This method may again be performed in any manner known per se, using techniques known to the person skilled in the art and/or as further described herein. For example, the selection, screening and isolation of the B-cells or immunoglobulin sequences may be performed using the so-called “Nanoclone™” technique, for which reference is made to the International application WO 06/079372 by Ablynx N.V.

In another aspect, a method for generating CDR nucleic acid sequences that bind to a serum protein, comprises at least the steps of:
a) providing a set, collection or library of nucleic acid sequences encoding CDR sequences; and
b) screening said set, collection or library of nucleic acid sequences for nucleic acid sequences that encode a CDR sequence that can bind to and/or have affinity for at least one domain or epitope of a serum protein; and
c) isolating said nucleic acid sequence.

This method may again be performed in a manner known per se, for which for example reference is made to WO 03/050531 (Ablynx N.V. and Algonomics N.V.).

Based on the sequence obtained in c) parts, analogs, fragments, variants can be prepared in a manner known per se, for example by site-specific mutagenesis (using mismatched primers), by de novo nucleic acid synthesis, or de novo peptide synthesis).

Alternatively, nucleic acid sequences encoding amino acid sequence of the invention (and in particular CDRs or suitable parts, analogs, fragments, variants thereof) can be generated by a method provided by the invention, at least comprising the steps of:
a) providing a set, collection or library of nucleic acid sequences encoding immunoglobulin sequences; and
b) screening said set, collection or library of nucleic acid sequences for nucleic acid sequences that encode an immunoglobulin sequence, respectively, that can bind to and/or have affinity for at least one domain or epitope of a serum protein; and
c) isolating said nucleic acid sequence.

This method may again be performed in a manner known per se, for which for example reference is made the techniques mentioned herein, as well as to WO 03/050531.

The nucleotide sequence obtained in step c) may again be expressed in order to provide an amino acid sequence of the invention, or may be converted (e.g. using one of the techniques cited herein) to provide a nucleic acid sequence that encodes a part, fragment, analog or variant of an amino acid sequence of the invention (which may then again be expressed in order to provide said part, fragment, variant or analog as an amino acid sequence).

Alternatively, said nucleic acid sequence or converted nucleic acid sequence may be linked (via a suitable spacer or linker) to any desired nucleic acid and subsequently be expressed (i.e. as a protein fusion); the obtained nucleic acid sequence may be for example (but not limited hereto) linked to a nucleic acid encoding a therapeutic moiety, as described herein, and subsequently be expressed as a polypeptide or protein construct or fusion protein.

In the above method, the set, collection or library of immunoglobulin sequences may be displayed on a phage, phagemid, ribosome or suitable micro-organism (such as yeast), such as to facilitate screening. Suitable methods, techniques and host organisms for displaying and screening (a set, collection or library of) immunoglobulin sequence or CDR sequences will be clear to the person skilled in the art, for example on the basis of the further disclosure herein.

In such a method, the set, collection or library of nucleic acid sequences encoding immunoglobulin sequences or CDR sequences may be a set, collection or library of nucleic acid sequences encoding a naïve set, collection or library of immunoglobulin sequences or CDR sequences; a set, collection or library of nucleic acid sequences encoding a synthetic or semi-synthetic set, collection or library of immunoglobulin sequences or CDR sequences; and/or a set, collection or library of nucleic acid sequences encoding a set, collection or library of immunoglobulin sequences or CDR sequences that have been subjected to affinity maturation.

Also, in such a method, the set, collection or library of nucleic acid sequences may encode a set, collection or library of (CDR sequences derived from) heavy chain variable domains or of (CDR sequences derived from) light chain variable domains. For example, the set, collection or library of nucleic acid sequences may encode a set, collection or library of (CDR sequences derived from) domain antibodies or single domain antibodies, or a set, collection or library of (CDR sequences derived from) immunoglobulin sequences that are capable of functioning as a domain antibody or single domain antibody.

In a preferred aspect of this method, the set, collection or library of nucleic acid sequences may encode an immune set, collection or library of (CDR sequences derived from) immunoglobulin sequences, for example derived from a mammal that has been suitably immunized with an antigen that comprises the desired extracellular part, region, domain, loop or other extracellular epitope(s), such that an immune response against the desired extracellular part, region, domain, loop or other extracellular epitope(s) is raised. In one specific, but non-limiting aspect, such a set, collection or library of nucleotide sequences may be derived from a Camelid.

The set, collection or library of nucleic acid sequences may encode may for example encode a immune set, collection or library of (CDR sequences derived from) heavy chain variable domains or of (CDR sequences derived from) light chain variable domains. In one specific aspect, the set, collection or library of nucleotide sequences may encode a set, collection or library of (CDR sequences derived from) VHH sequences.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding immunoglobulin sequences will be clear to the person skilled in the art, for example on the basis of the further disclosure herein. Reference is also made to the review by Hoogenboom in Nature Biotechnology, 23, 9, 1105-1116 (2005).

The invention also relates to amino acid sequences or nucleic acid sequences of the invention that are obtained by the above methods.

The amino acid sequences disclosed herein can be used with advantage as a fusion partner in order to increase the half-life of therapeutic moieties such as proteins, compounds (including, without limitation, small molecules) or other therapeutic entities.

Thus, in another aspect, the invention provides polypeptide or protein constructs that comprise or essentially consist of an amino acid sequence as disclosed herein. In particular, the invention provides polypeptide or protein constructs that comprise or essentially consist of at least one amino acid sequence of the invention that is linked to at least one therapeutic moiety, optionally via one or more suitable linkers or spacers. Such polypeptide or protein construct may for example (without limitation) be a fusion protein, as further described herein.

The invention further relates to therapeutic uses of polypeptide or protein constructs or fusion proteins and to pharmaceutical compositions comprising such polypeptide or protein constructs or fusion proteins.

In some embodiments the at least one therapeutic moiety comprises or essentially consists of a therapeutic protein, polypeptide, compound, factor or other entity. In a preferred embodiment the therapeutic moiety is directed against a desired antigen or target, is capable of binding to a desired antigen (and in particular capable of specifically binding to a desired antigen), and/or is capable of interacting with a desired target. In another embodiment, the at least one therapeutic moiety comprises or essentially consists of a therapeutic protein or polypeptide. In a further embodiment, the at least one therapeutic moiety comprises or essentially consists of an immunoglobulin or immunoglobulin sequence (including but not limited to a fragment of an immunoglobulin), such as an antibody or an antibody fragment (including but not limited to an ScFv fragment). In yet another embodiment, the at least one therapeutic moiety comprises or essentially consists of an antibody variable domain, such as a heavy chain variable domain or a light chain variable domain.

In a preferred embodiment, the at least one therapeutic moiety comprises or essentially consists of at least one domain antibody or single domain antibody, “dAb” or Nanobody®. According to a preferred aspect of this embodiment, the amino acid sequence of the invention preferably comprises or essentially consists of a CDR (such as a CDR3 loop) derived from a domain antibody or single domain antibody, “dAb” or Nanobody®, so that the resulting polypeptide or protein construct or fusion protein is a multivalent construct and preferably a multispecific construct comprising or essentially consisting of at least one domain antibody, single domain antibody, “dAb” or Nanobody® (or a combination thereof), linked to (optionally via one or more suitable linkers) to at least one CDR (such as a CDR3 loop) derived from a domain antibody, single domain antibody, “dAb” or Nanobody®, which binds to a serum protein.

By a “multivalent” compound, protein, polypeptide or construct is in particular meant in this description a compound, protein, polypeptide or construct that comprises at least two binding units (i.e. binding to the same or different epitopes), both of which can bind to the same biological molecule. By a “bivalent” compound, protein, polypeptide or construct is meant in this description, a compound, protein, polypeptide or construct that comprises two binding units, which can bind to the same biological molecule. By a “monovalent” compound, protein or polypeptide is meant in this description, a compound, protein or polypeptide that essentially consists of one binding unit, which can bind to a biological molecule.

By “binding unit” is in particular meant in this description any amino acid sequence, peptide, protein, polypeptide, construct, fusion protein, compound, factor or other entity capable of binding a biological molecule as described herein, such as an amino acid sequence of the invention or a therapeutic moiety (both as described herein). When a compound, protein, polypeptide or construct comprises two or more binding units, said binding units may optionally be linked to each other via one or more suitable linkers.

By a “multispecific” compound, protein, polypeptide or construct is in particular meant in this description, a compound, protein, polypeptide or construct that comprises at least two binding units, of which at least a first binding unit can bind to a first biologically functional molecule and of which at least a second binding unit can bind to a second biologically functional molecule. By a “bispecific” compound, protein, polypeptide or construct is meant in this description, a compound, protein, polypeptide or construct that comprises two binding unit, of which the first binding unit can bind to a first biologically functional molecule and of which the second binding unit can bind to a second biologically functional molecule.

In a specific embodiment, the at least one therapeutic moiety comprises or essentially consists of at least one monovalent Nanobody® or a bivalent, multivalent, bispecific or multispecific Nanobody® construct. According to this embodiment, the amino acid sequence of the invention, linked to said therapeutic moiety, preferably comprises or essentially consists of at least one CDR (such as a CDR3 loop) derived from a domain antibody, single domain antibody or “dAb”, and more preferably comprises or essentially consists of a CDR (such as a CDR3 loop) derived from a Nanobody®, so that the resulting construct or fusion protein is a multivalent construct or fusion protein (as defined herein) and preferably a multispecific construct or fusion protein (as defined herein) comprising at least one Nanobody® and at least one CDR derived from a domain antibody, single domain antibody or “dAb” and more preferably at least one CDR derived from a Nanobody®, which binds to a serum protein.

When the amino acid sequences, compounds, proteins, polypeptides, fusion proteins, or multivalent or multispecific constructs of the invention are intended for pharmaceutical or diagnostic use, they preferably bind to a human serum protein. According to one preferred, but non-limiting embodiment the amino acid sequences, compounds, proteins, polypeptides, fusion proteins, or multivalent or multispecific constructs of the invention show an affinity for a human serum protein that is higher than the affinity for a mouse serum protein.

Non-limiting examples of serum proteins to which the amino acid sequences, compounds, proteins, polypeptides, fusion proteins, or multivalent or multispecific constructs of the invention can bind to are serum albumin, serum immunoglobulins, thyroxine-binding protein, transferrin or fibrinogen; preferably the amino acid sequences, peptides, proteins, polypeptides, constructs, fusion proteins of the invention bind to serum albumin, and more preferably to human serum albumin.

Generally, the protein or polypeptide constructs or fusion proteins of the invention, comprising or essentially consisting of at least one amino acid sequence of the invention that is linked to at least one therapeutic moiety (optionally via one or more suitable linkers or spacers) preferably have a half-life that is at least 1.5 times, preferably at least 2 times, such as at least 5 times, for example at least 10 times or more than 20 times, greater than the half-life of the corresponding therapeutic moiety per se.

Also, preferably, any such protein or polypeptide construct or fusion protein has a half-life that is increased with more than 1 hour, preferably more than 2 hours, more preferably of more than 6 hours, such as of more than 12 hours, compared to the half-life of the corresponding therapeutic moiety per se.

Also, preferably, any such protein or polypeptide construct or fusion protein has a half-life that is more than 1 hour, preferably more than 2 hours, more preferably of more than 6 hours, such as of more than 12 hours, and for example of about one day, two days, one week, two weeks or three weeks, and preferably no more than 2 months, although the latter may be less critical.

Also, as mentioned above, when the amino acid sequence of the invention comprises or essentially consists of a CDR3 sequence derived from a domain antibody, single domain antibody, “dAb” or preferably derived from a Nanobody®, it can be used to increase the half-life of other immunoglobulin sequences, such as domain antibodies, single domain antibodies, “dAb's” or preferably of Nanobodies®.

Thus, one embodiment of the invention relates to a protein or polypeptide construct or fusion protein that comprises or essentially consists of at least one amino acid sequence of the invention and at least one immunoglobulin sequence, such as a domain antibody, a single domain antibody, a “dAb” or a Nanobody®. The immunoglobulin sequence is preferably directed against a desired target (which is preferably a therapeutic target), and/or another immunoglobulin sequence that is useful or suitable for therapeutic, prophylactic and/or diagnostic purposes.

Thus, in another aspect, the invention relates to a multispecific (and in particular bispecific) construct that comprises or essentially consists of at least one CDR sequence (such as a CDR3 sequence) and at least one Nanobody®, in which said at least one Nanobody® is preferably directed against a desired target (which is preferably a therapeutic target), and/or another Nanobody® that is useful or suitable for therapeutic, prophylactic and/or diagnostic purposes.

The invention also relates to nucleotide sequences or nucleic acids that encode amino acid sequences, compounds, proteins, polypeptides, fusion proteins, or multivalent or multispecific constructs described herein. The invention further includes genetic constructs that include the foregoing nucleotide sequences or nucleic acids and one or more elements for genetic constructs known per se. The genetic construct may be in the form of a plasmid or vector. Such and other genetic constructs are known by those skilled in the art.

The invention also relates to hosts or host cells that contain such nucleotide sequences or nucleic acids, and/or that express (or are capable of expressing) amino acid sequences, compounds, proteins, polypeptides, fusion proteins, or multivalent or multispecific constructs described herein. Again, such hosts or host cells are known by those skilled in the art.

The invention also generally relates to a method for preparing amino acid sequences, compounds, proteins, polypeptides, fusion proteins, or multivalent or multispecific constructs as described herein, which method comprises cultivating or maintaining a host cell as described herein under conditions such that said host cell produces or expresses an amino acid sequence, compound, protein, polypeptide, fusion protein, or multivalent or multispecific construct as described herein, and optionally further comprises isolating the amino acid sequence, compound, protein, polypeptide, fusion protein, or multivalent or multispecific construct so produced. Again, such methods can be performed as generally described in the co-pending patent applications by Ablynx N.V. described herein, such as WO 04/041862 or WO 06/122825.

The invention also encompasses medical uses and methods of treatment encompassing the amino acid sequence, compound, or multivalent and multispecific compound of the invention, wherein said medical use or method is characterized in that said medicament is suitable for administration at intervals of at least about 50% of the natural half-life of human serum albumin.

The invention also relates to methods for extending or increasing the serum half-life of a therapeutic (i.e. a therapeutic moiety, compound, protein or other therapeutic entity). The methods include contacting the therapeutic with any of the foregoing amino acid sequences, such that the therapeutic is bound to or otherwise associated with the amino acid sequences, compounds, fusion proteins or constructs of the invention. In some embodiments, the therapeutic is a biological therapeutic, preferably a peptide or a polypeptide, in which case the step of contacting the therapeutic can include preparing a fusion protein by linking the peptide or polypeptide with the amino acid sequence, compound, fusion proteins or constructs of the invention.

These methods can further include administering the therapeutic to a subject after the therapeutic is bound to or associated with the amino acid sequence, compound, fusion protein or construct of the invention. In such methods, the serum half-life of the therapeutic is at least 1.5 times the half-life of therapeutic per se, or is increased by at least 1 hour compared to the half-life of therapeutic per se. In some preferred embodiments, the serum half-life of the therapeutic is at least 2 times, at least 5 times, at least 10 times, or more than 20 times greater than the half-life of the corresponding therapeutic moiety per se. In other preferred embodiments, the serum half-life of the therapeutic is increased by more than 2 hours, more than 6 hours or more than 12 hours compared to the half-life of the corresponding therapeutic moiety per se.

In another aspect, the invention relates to a method for modifying a therapeutic such that the desired therapeutic level of said therapeutic is, upon suitable administration of said therapeutic so as to achieve said desired therapeutic level, maintained for a prolonged period of time.

The methods include contacting the therapeutic with any of the foregoing amino acid sequences, such that the therapeutic is bound to or otherwise associated with the amino acid sequences, compounds, fusion proteins or constructs of the invention. In some embodiments, the therapeutic is a biological therapeutic, preferably a peptide or polypeptide, in which case the step of contacting the therapeutic can include preparing a fusion protein by linking the peptide or polypeptide with the amino acid sequence, compound, fusion protein, or constructs of the invention.

These methods can further include administering the therapeutic to a subject after the therapeutic is bound to or otherwise associated with the amino acid sequence, compound, fusion protein, or construct of the invention, such that the desired therapeutic level is achieve upon such administration. In such methods, the time that the desired therapeutic level of said therapeutic is maintained upon such administration is at least 1.5 times the half-life of therapeutic per se, or is increased by at least 1 hour compared to the half-life of therapeutic per se. In some preferred embodiments, the time that the desired therapeutic level of said therapeutic is maintained upon such administration is at least 2 times, at least 5 times, at least 10 times or more than 20 times greater than the half-life of the corresponding therapeutic moiety per se. In other preferred embodiments, the time that the desired therapeutic level of said therapeutic is maintained upon such administration is increased by more than 2 hours, more than 6 hours or more than 12 hours compared to the half-life of the corresponding therapeutic moiety per se.

Preferably, the time that the desired therapeutic level of said therapeutic is maintained upon such administration is increased such that the therapeutic can be administered at a frequency that is as defined herein for the compounds of the invention.

In another aspect, the invention relates to the use of a compound of the invention (as defined herein) for the production of a medicament that increases and/or extends the level of the therapeutic agent in said compound or construct in the serum of a patient such that said therapeutic agent in said compound or construct is capable of being administered at a lower dose as compared to the therapeutic agent alone (i.e. at essentially the same frequency of administration).

The invention also relates to a pharmaceutical composition that comprises at least one amino acid sequence, compound, protein, polypeptide, fusion protein, or multivalent or multispecific construct as described herein, and optionally at least one pharmaceutically acceptable carrier, diluent or excipient. Such preparations, carriers, excipients and diluents may generally be as described in the co-pending patent applications by Ablynx N.V. described herein, such as WO 04/041862 or WO 06/122825.

However, since the amino acid sequences, compounds, proteins, polypeptides, fusion proteins, or multivalent or multispecific constructs described herein have an increased half-life, they are preferably administered to the circulation. As such, they can be administered in any suitable manner that allows the amino acid sequences, compounds, proteins, polypeptides, fusion proteins, or multivalent or multispecific constructs to enter the circulation, such as intravenously, via injection or infusion, or in any other suitable manner (including oral administration, administration through the skin, intranasal administration, administration via the lungs, etc). Suitable methods and routes of administration will be clear to the skilled person, again for example also from the teaching of WO 04/041862 or WO 06/122825.

Thus, in another aspect, the invention relates to a method for the prevention and/or treatment of at least one disease or disorder that can be prevented or treated by the use of amino acid sequences, compounds, proteins, polypeptides, fusion proteins, or multivalent or multispecific constructs described herein, which method comprises administering, to a subject in need thereof, a pharmaceutically active amount of a amino acid sequences, compounds, proteins, polypeptides, fusion proteins, or multivalent or multispecific constructs of the invention, and/or of a pharmaceutical composition comprising the same. As will be clear to the skilled person, the diseases and disorders that can be prevented or treated by the use of amino acid sequences, compounds, proteins, polypeptides, fusion proteins, or multivalent or multispecific constructs described herein will generally be the same as the diseases and disorders that can be prevented or treated by the use of the therapeutic moiety that is present in the amino acid sequences, compounds, proteins, polypeptides, fusion proteins, or multivalent or multispecific constructs of the invention.

In the context of the present invention, the term “prevention and/or treatment” not only comprises preventing and/or treating a disease, but also generally comprises preventing the onset of a disease, slowing or reversing the progress of a disease, preventing or slowing the onset of one or more symptoms associated with a disease, reducing and/or alleviating one or more symptoms associated with a disease, reducing the severity and/or the duration of a disease and/or of any symptoms associated therewith and/or preventing a further increase in the severity of a disease and/or of any symptoms associated therewith, preventing, reducing or reversing any physiological damage caused by a disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

More specifically, the present invention relates to a method of treatment wherein the frequency of administering the amino acid sequence, compound, fusion protein or construct of the invention is at least 50% of the natural half-life of serum albumin in said mammal (i.e. in the case of man, of human serum albumin), preferably at least 60%, preferably at least 70%, more preferably at least 80%, and most preferably at least 90%.

Specific frequencies of administration to a mammal, which are within the scope of the present invention are at least 50%, 55%, 60%, 65%, 70%, 75%, 80%, 85%, 90%, 95%, or at least 100% of the natural half-life of serum albumin in said mammal as defined above.

In other words, specific frequencies of administration, which are within the scope of the present invention are every 4, 5, 6, 7, 8, 9, 10, 11, 12, 13, 14, 15, 16, 17, 18, or 19 days.

Without limitation, the frequencies of administration referred to above are in particular suited for maintaining a desired level of the amino acid sequence, compound, fusion protein or construct in the serum of the subject treated with the amino acid sequence, compound, fusion protein, or construct, optionally after administration of one or more (initial) doses that are intended to establish said desired serum level. As will be clear to the skilled person, the desired serum level may inter alia be dependent on the amino acid sequence, compound, fusion protein, or construct used and/or the disease to be treated. The clinician or physician will be able to select the desired serum level and to select the dose(s) and/or amount(s) to be administered to the subject to be treated in order to achieve and/or maintain the desired serum level in said subject, when the amino acid sequence, compound, fusion protein, or construct of the invention is administered at the frequencies mentioned herein.

In another embodiment, the invention relates to a method for immunotherapy, and in particular for passive immunotherapy, which method comprises administering, to a subject suffering from or at risk of the diseases and disorders mentioned herein, a pharmaceutically active amount of a fusion protein or construct of the invention, and/or of a pharmaceutical composition comprising the same.

The amino acid sequences, compounds, proteins, polypeptides, fusion proteins, or multivalent or multispecific constructs and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific amino acid sequence, compound, protein, polypeptide, fusion protein, or multivalent or multispecific construct of the invention to be used, the specific route of administration and pharmaceutical formulation or composition to be used, the age, gender, weight, diet, general condition of the patient, and similar factors well known to the clinician.

Generally, the treatment regimen will comprise the administration of one or more amino acid sequences, compounds, proteins, polypeptides, fusion proteins, or multivalent or multispecific constructs of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of intended diseases and disorders (i.e. those diseases and disorders which are usually treated or prevented through the use of the therapeutic entity per se) and depending on the specific disease or disorder to be treated, the potency and/or the half-life of the specific amino acid sequences, compounds, proteins, polypeptides, fusion proteins, or multivalent or multispecific constructs to be used, the specific route of administration and the specific pharmaceutical formulation or composition used, the amino acid sequences, compounds, proteins, polypeptides, fusion proteins, or multivalent or multispecific constructs of the invention will generally be administered in an amount between 1 gram and 0.01 microgram per kg body weight per day, preferably between 0.1 gram and 0.1 microgram per kg body weight per day, such as about 1, 10, 100, 1000, or 2000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single amino acid sequence, compound, protein, polypeptide, fusion protein, or multivalent or multispecific construct of the invention will be used. It is however within the scope of the invention to use two or more amino acid sequences, compounds, proteins, polypeptides, fusion proteins, or multivalent or multispecific constructs of the invention in combination.

The amino acid sequences, compounds, proteins, polypeptides, fusion proteins, or multivalent or multispecific constructs of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

In particular, the amino acid sequences, compounds, proteins, polypeptides, fusion proteins, or multivalent or multispecific constructs of the invention may be used in combination with other pharmaceutically active compounds or principles that are or can be used for the prevention and/or treatment of the diseases and disorders that can be prevented or treated with the amino acid sequences, compounds, proteins, polypeptides, fusion proteins, or multivalent or multispecific constructs of the invention, and as a result of which a synergistic effect may or may not be obtained.

The effectiveness of the treatment regimen used according to the invention may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

### BRIEF DESCRIPTION OF THE DRAWINGS

The invention will be further illustrated by means of the following non-limiting Experimental part and Figures. The non-limiting Figures show:

FIG. 1: Sequence of oligonucleotides for construction of constrained and non-constrained CDR3 library. IUPAC codes are used. The oligonucleotide primers shown are: For1Sfi (SEQ ID NO:7); For2Sfi (SEQ ID NO:8); For3Sfi (SEQ ID NO:9); For4Sfi (SEQ ID NO:10); For5Sfi (SEQ ID NO:11); For6Sfi (SEQ ID NO:12); For7Sfi (SEQ ID NO:13); Back1Not (SEQ ID NO:14); Back2Not (SEQ ID NO:15); Back3Not (SEQ ID NO:16); Back4Not (SEQ ID NO:17); Back5Not (SEQ ID NO:18); Back1cysRNot (SEQ ID NO:19); Back1cysWNot (SEQ ID NO:20); Back2cysWNot (SEQ ID NO:21); Back3cysWNot (SEQ ID NO:22); Back3cysRNot (SEQ ID NO:23); Back4cysWNot (SEQ ID NO:24); Back5cysWNot (SEQ ID NO:25).

FIG. 2: Immune response of llamas 117 and 118 to: a) human serum albumin (FIG. 2A), b) mouse serum albumin (FIG. 2B), c) cynomolgus serum albumin (FIG. 2C) and d) baboon serum albumin (FIG. 2D).

FIG. 3: Odyssey read-out of screening of monoclonal phages after two selection rounds on HSA. Odd and even columns are coated with HSA and ovalbumin respectively. The two boxes indicate two different monoclonal phage that bind to HSA but not to ovalbumin, although the signal/background ratio is very low. Sequencing revealed that both CDR3 loops are identical: dtavyycnaaasysdydvfgggtdfgpwgqgtqv (SEQ ID NO:1; flanking FR sequence in italic). This peptide is referred to as 17D12.

FIG. 4: Amino acid sequence of CDR3 loop of 17D12 with or without flanking FR expressed on pIII of M13 phage. FR residues flanking the CDR3 of 17D12 (SEQ ID NO:1), as selected from primary screening after 2 selection rounds on HSA, are in italic. Residues added to the CDR3 of 17D12 to construct non-constrained (NC; SEQ ID NO:26) and constrained (C; SEQ ID NO:27) truncated peptides, are in bold.

FIG. 5: Binding of CDR3 loop of 17D12 with or without flanking FR expressed on pIII of M13 phage to HSA. Full length 17D12 (), as well as the CDR3 loop without flanking FR residues in non-constrained and constrained format, i.e. 17D12-CDR3-NC (▴) or 17D12-CDR3-C () respectively, bind dose-dependently to HSA (full line), but not to negative control peptide, ovalbumin (dotted line).

FIG. 6: Cross-reactivity and specificity of CDR3 loop of 17D12 with or without flanking FR expressed on pIII of M13 phage. Full length 17D12, as well as the CDR3 loop without flanking FR residues in non-constrained and constrained format, i.e. 17D12-CDR3-NC or 17D12-CDR3-C respectively, bind dose-dependently to HSA and CSA. Some non-specific binding of 17D12-CDR3-C to irrelevant proteins ovalbumin and TNF is present. -: non-coated.

FIG. 7: Surface plasmon resonance analysis of the binding of human serum albumin to synthetic peptides PEO-100 and PEO-101.5. Panel a: PEO-105 (Ala-17D12-CDR3); panel b: PEO-101.5 (Cys-Ala-17D12-CDR3).

FIG. 8: Amino acid sequence of VHH-17D12 fusions expressed on pIII of M13 phage. FR residues of 17D12 are in italic. The Cys residue in FR3 was replaced with a Ser. VHH-17D12(S), SEQ ID NO: 31; VHH-GlySer-17D12(S), SEQ ID NO: 32.

FIG. 9: Binding of VHH-17D12 fusions expressed on pIII of M13 phage to HSA. Full length 17D12 (), as well as 17D12 peptide fused at C-terminus of VHH 2D3, with specificity to HER2, with or without Gly4Ser-Gly3Ser linker, i.e. 2D3-GlySer-17D12(S) () and 2D3-17D12(S) (▴) respectively, bind dose-dependently to HSA (full line), but not to negative control peptide, ovalbumin (dotted line).

FIG. 10: Surface plasmon resonance analysis of the binding of Nanobody® (2D3) and Nanobody® fusion peptide (2D3-17D12) on human serum albumin (HSA). Coating of the chip (CM5) was performed by amine coupling using NHS/EDC for activation and ethanolamine for deactivation (Biacore amine coupling kit). hip coated with 7000 RU human serum albumin (Sigma, 99% pure) and 2500 RU irrelevant protein antigen. 2D3 and 2D3-17D12 was successively injected over the chip in increasing concentrations (1.25 μM, 2.5 μM and 5 μM). HBS-EP was used as flow buffer at a rate of 10 μl min-1.20 μl of sample was injected for 20 s.

FIG. 11: Surface plasmon resonance analysis of simultaneous binding of 2D3-17D12 fusion protein to HER2 antigen and human serum albumin (HSA). 2D3-17D12 fusion protein was pre-incubated or not with 1 and 5 μM HSA, followed by flow over immobilized 2D3 target antigen, rhErbB2-Fc. The dotted line represents the binding characteristics of 2D3 in presence of 25 μM HSA.

### EXPERIMENTAL PART

### Example 1

### Immunizations

### 1.1 Immunizations

After approval of the Ethical Committee of the Faculty of Veterinary Medicine (University Ghent, Belgium), llamas were immunized, according to standard protocols, with 6 intramuscular injections at weekly intervals of the following antigen cocktails:
Llama 006: human serum albumin (HSA) in cocktail with mouse TNF and IFN gamma
Llama 021: mouse serum albumin (MSA) in cocktail with PDK-1, Anti-CD4, hTNFalpha, Collagen type III+boosted with cynomolgus monkey albumin, human serum albumin
Llama 022: MSA in cocktail with PDK-1, hIFN gamma, hTNFalpha, Collagen type III
Llama 039: HSA, MSA, MSA, MonkeySA, MonkeySA, HSA
Llama 117: alternate immunization with HSA and a cocktail of BaboonSA, CynomolgusSA & MSA
Llama 118: alternate immunization with HSA and a cocktail of BaboonSA, CynomolgusSA & MSA

### 1.2. Evaluation of Induced Responses in Llama.

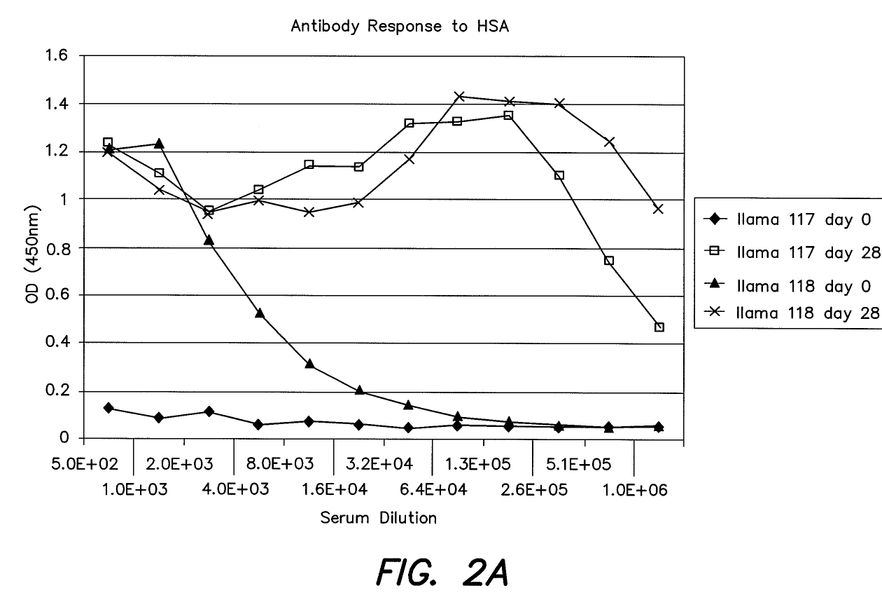

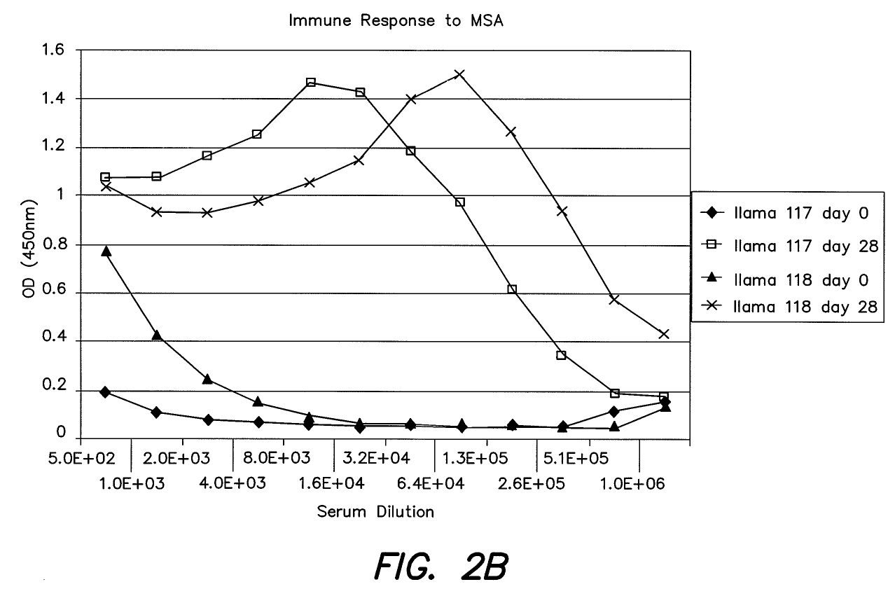

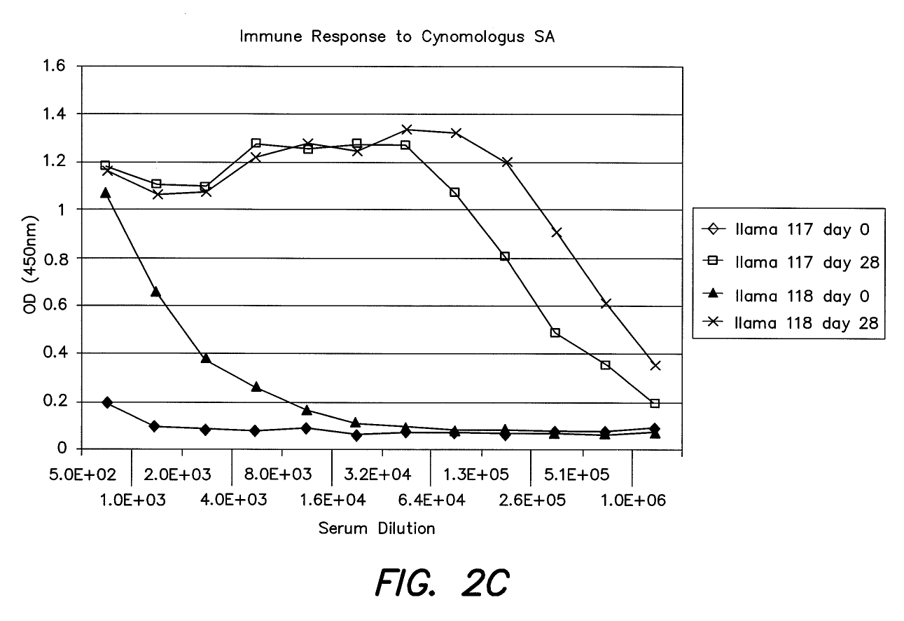

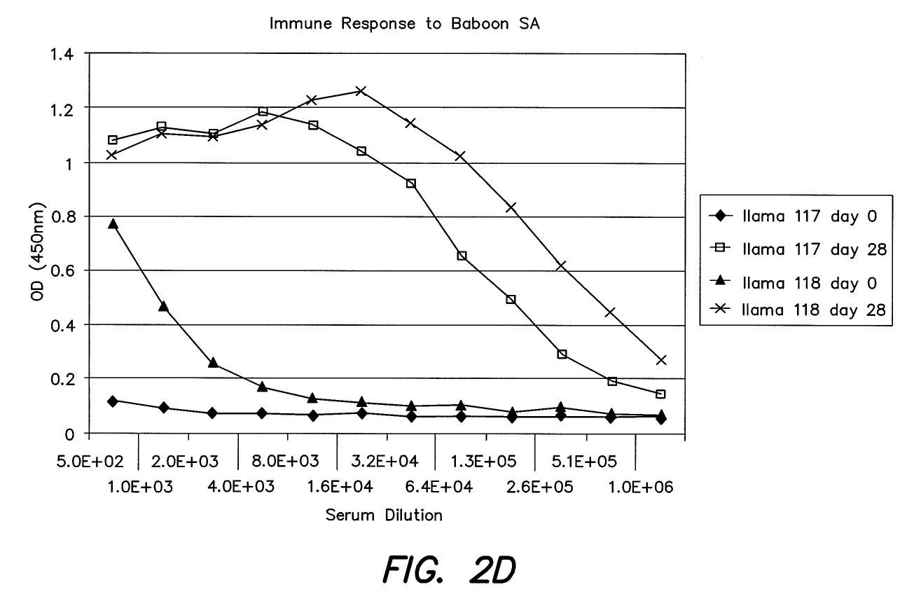

At day 0, 28 and time of PBL collection, sera were collected from llama's 117 and 118 to evaluate the induction of immune responses in the animals against albumin by ELISA. In short, 2 μg/ml of HSA, MSA, cynoSA or baboonSA were immobilized overnight at 4° C. in a 96 well Maxisorp plate (Nunc). Wells were blocked with a casein solution (1% in PBS). After addition of serum dilutions, specifically bound immunoglobulins were detected using a goat anti-llama horseradish peroxidase conjugate (Bethyl Lab. Inc.), showing that for both animals a significant antibody dependent immune response against human (FIG. 2A) mouse (FIG. 2B), cyno (FIG. 2C) and baboon (FIG. 2D) albumin was induced. The preimmune serum from llama 118 showed binding to the respective albumins up to a dilution of 1:10.000 but was clearly excelled by serum taken after 3 immunizations with albumin. No binding of the preimmune serum of llama 117 to the albumin of different species origin was detected.

### Example 2

### Library Construction

To identify CDR3 sequences which can bind albumin out of the context of the originating VHH template, two approaches are followed. In a first approach, populations of CDR3 loops are anchored on a microscaffold restraining the base of the loop. In VHH, and VH, the CDR3 loop is anchored on FR3 and FR4. As these regions are extended structures implied in an anti-parallel beta sheet organization, it is somehow obvious to people skilled in the art to include the last part of the end of FR3 and FR4 in the scaffold. In order to further restrain the base of the CDR3 loop, a non-natural disulphide bridge was engineered between position 93 in FR3 (Kabat numbering), a conserved Cys residue and position 104 (Kabat numbering), a conserved Gly residue.

The complementary determining region 3 (CDR3) of VHHs isolated from lymph nodes (LN) or peripheral blood lymphocytes (PBL) of immunized llamas were expressed on the surface of M13 bacteriophages as N-terminal fusion to geneIII protein with flanking framework (FR) residues. More specifically, the library construct contains a pIII secretion signal peptide, the CDR3 library flanked with 9 FR3 and 7 FR4 residues, followed by His₆ and c-myc tags respectively, a short Gly-Ala-Ala linker and the M13 pIII gene. Next to this non-constrained library, due to the conserved Cys in FR3, a constrained library construct was designed by replacing the first residue in FR4 (Trp or Arg) by Cys.

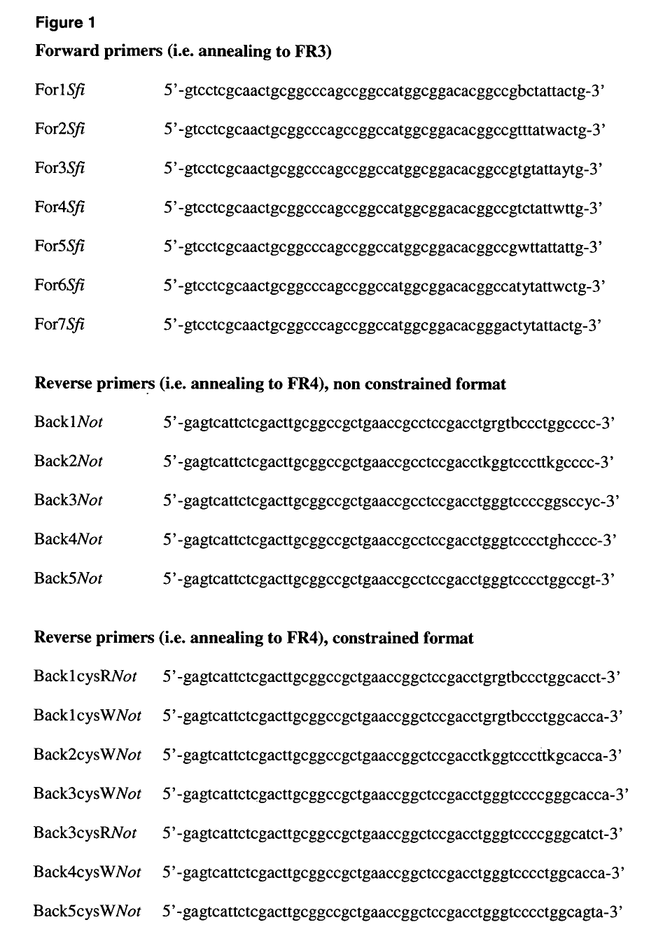

The two described CDR3 libraries were constructed as follows. RNA was isolated from lymph node and/or peripheral blood lymphocytes of immunized llama's, followed by cDNA synthesis using random hexamers and Superscript III, according to the manufacturer's instructions (Invitrogen). In a first PCR the VHH and VH were amplified using a forward primer mix [4:1 ratio of ABL051 (5′-ggctgagctgggtggtcctgg-3′, SEQ ID NO:4) and ABL052 (5′-ggctgagtttggtggtcctgg-3′, SEQ ID NO:5) respectively] and reverse primer ABL003 (5′-ggtacgtgctgttgaactgttcc-3′, SEQ ID NO:6). After isolation of the VHH fragment, two separate nested PCRs were performed to amplify CDR3 with flanking FR sequence in a non-constrained or constrained format. In both nested PCRs a mix of 7 degenerated forward primers (i.e. annealing to FR3) was used (SEQ ID NO's: 7 to 13, respectively), combined with a mix of 5 (SEQ ID NO's: 14 to 18) or 7 (SEQ ID NO's: 19 to 25) reverse primers (i.e. annealing to FR4) for the non-constrained and constrained format respectively (see FIG. 1, IUPAC codes are used.). The nested PCR-fragments were cloned upstream of the pIII gene via the SfiI and NotI restriction sites in the in-house constructed, pUC19-derived, pAX50 vector. From each immunized animal, two libraries were obtained of ˜5×10⁷.

### Example 3

### Selection and Screening

Selections were performed on human serum albumin (HSA: A5763, Sigma). Wells of a Maxisorp microtiter plate (Nunc) were coated using 10 μg/mL HSA and blocked with SuperBlock T20 PBS Blocking Buffer (Pierce) to prevent non-specific binding. Before adding the phages to the coated wells, they were pre-incubated in 1:1 (v:v) Superblock T20 PBS Blocking Buffer:PBS+0.05% Tween-20. Phages were eluted with 10mM triethylamine and neutralized with 1M Tris pH 7.5.

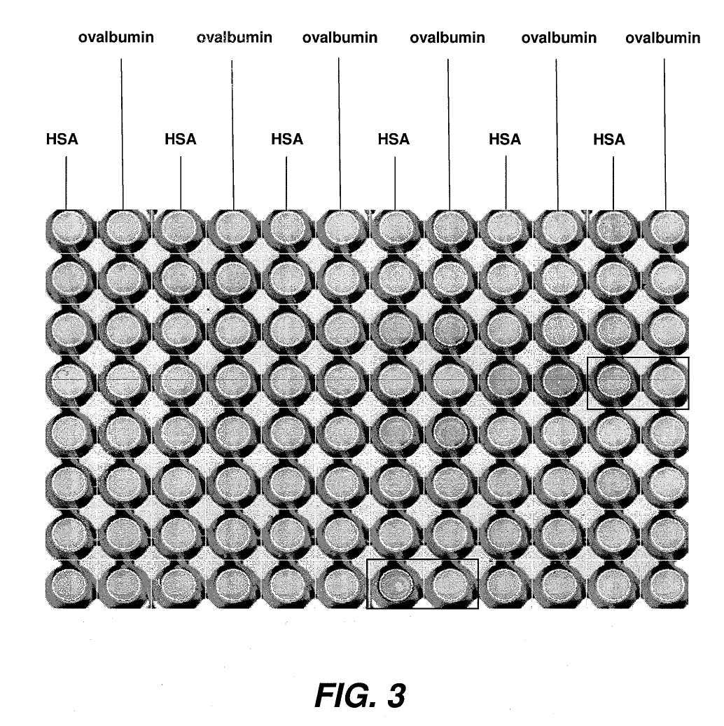

After two selection rounds, monoclonal phage were screened for binding on HSA versus negative control antigen, ovalbumin (A5378, Sigma). Wells of a Maxisorp microtiter plate (Nunc) were coated using 10 μg/mL HSA or ovalbumin and blocked with SuperBlock T20 PBS Blocking Buffer (Pierce) to prevent non-specific binding. Phages, produced in 96-well plate, were diluted 1/10 in 1:1 (v:v) Superblock T20 PBS Blocking Buffer:PBS+0.05% Tween-20 and incubated on both HSA and ovalbumin. Bound phages were detected using anti-M13 (GE Healthcare) and goat anti-mouse IRDye700 (Rockland) and signals were read-out on Odyssey (LI-COR Biosciences). FIG. 3 shows an example of a screening read-out where two HSA-binding CDR3 loops were identified. Both ‘hits’ were selected from the same library, i.e. originating from llama 117 in the non-constrained format. Sequencing revealed that both identified CDR3 loops are identical: dtavyycnaaasysdydvfgggtdfgpwgqgtqv (flanking FR sequences in italics). This peptide is referred to as 17D12. Its full amino acid sequence is given in SEQ ID NO: 1, and its encoding nucleotide sequence is given in SEQ ID NO:2. The amino acid sequence of the CDR3 loop is given in SEQ ID NO:3.

### Example 4

### Binding of CDR3 Loop of 17D12 with or without Flanking FR Expressed on pIII of M13 Phage to HSA

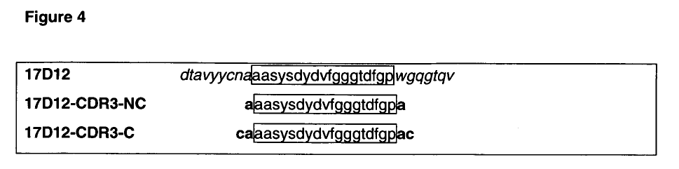

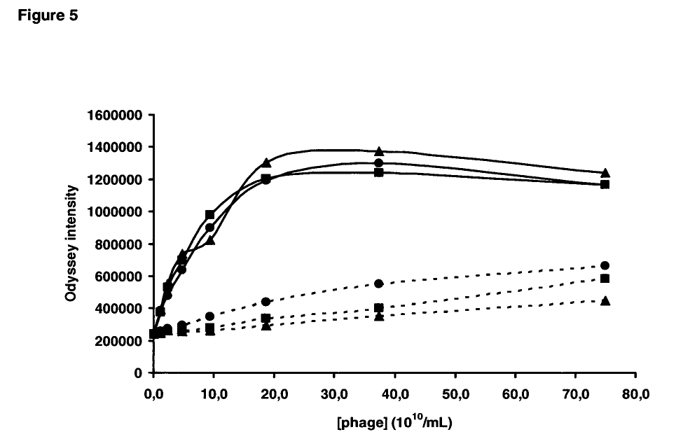

Binding of 17D12, as selected from the primary screening, to HSA and negative control peptide, ovalbumin, was assessed using different phage concentrations. Additionally, binding of two truncated versions of 17D12 was analyzed. Both truncated peptides, i.e. 17D12-CDR3-NC (SEQ ID NO: 26) and 17D12-CDR3-C (SEQ ID NO:27), lack the flanking FR residues and the former is a non-constrained loop, whereas the latter is constrained (FIG. 4).

The phage binding assay was performed essentially as described in example 3. Phages, produced in 10 mL culture volume, were incubated on both HSA and ovalbumin in a ½ dilution series, starting with 7.5×10¹¹ phages/mL. FIG. 4 shows dose-dependent binding of both the full length 17D12 peptide as well as the truncated peptides, lacking the FR residues, to HSA. Non-specific binding to ovalbumin is slightly higher for the constrained truncated peptide.

### Example 5

### Cross-Reactivity and Specificity of CDR3 Loop of 17D12 with or without Flanking FR Expressed on pIII of M13 Phage

Binding of 17D12, as selected from primary screening after 2 selection rounds on HSA, to serum albumin of different species [mouse serum albumin (MSA, A3559, Sigma), cynomolgus serum albumin (CSA, in-house production), bovine serum albumin (BSA, A6003, Sigma) and HSA] and to negative control antigens [ovalbumin, human tumor necrosis factor α (TNF, in-house production)] was assessed using different phage concentrations. Additionally, similar binding studies were performed with two truncated variants of 17D12. Both peptides, i.e. 17D12-CDR3-NC (SEQ ID NO: 26) and 17D12-CDR3-C (SEQ ID NO;27), lack the flanking FR residues of 17D12 and the former is a non-constrained loop, whereas the latter is constrained (FIG. 4).

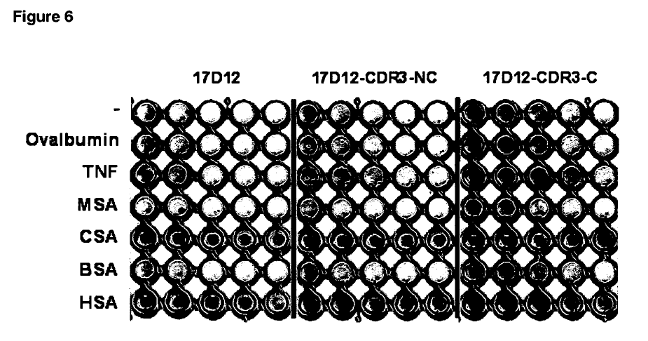

The phage binding assay was performed essentially as described in example 4. Full length 17D12 peptide as well as truncated peptides, 17D12-CDR3-NC and 17D12-CDR3-C, bind dose-dependently to HSA and CSA, whereas reactivity to MSA and BSA was not apparent in the assay performed (FIG. 6). There is no significant non-specific binding of 17D12 and 17D12-CDR3-NC to irrelevant proteins tested (ovalbumin and TNF). In contrast, there is a subtle degree of non-specific binding with the constrained truncated peptide.

### Example 6

### Binding of Synthetic Peptide to HSA

Peptides shown in Table 1 were synthesized and purified by Pepscan Presto (Lelystad, The Netherlands) as follows. The peptides were synthesized by resin-based Fmoc (9-fluorenylmethoxycarbonyl) chemistry using a multiple peptide synthesizer. Biotin NovaTag resin (Novabiochem) was used and the peptide sequence was assembled according to the concept of Solid Phase Peptide Synthesis (SPPS). The peptides were purified by electrospray mass spectrometry driven preparative RP-HPLC, and purity and mass were checked by analytical RP-HPLC and electrospray mass spectrometry and determined to be 94.07% (acetyl-AAASYSDYDVFGGGTDFGP-c2 linker-biotin, SEQ ID NO:28), and 82.59% (acetyl-CAAASYSDYDVFGGGTDFGP-c2 linker-biotin, SEQ ID NO:29).

TABLE 1

Synthetic, biotinylated peptides containing
CDR3 sequence of 17D12 proceeded by Ala or
Cys-Ala.

| | |
|---|---|
| Ala-17D12-CDR3 | acetyl-AAASYSDYDVFGGGT |
| | DFGP-c2 linker-biotin |
| Cys-Ala-17D12-CDR3 | acetyl-CAAASYSDYDVFGGG |
| | TDFGP-c2 linker-biotin |

Binding of the 2 synthetic peptides Ala-17D12-CDR3 and Cys-Ala-17D12-CDR3 to HSA was assessed by surface plasmon resonance. The biotinylated peptides are each captured on streptavidin-coated sensorchip SA T071122. HSA binding is assessed at various concentrations. The samples were injected for 4 min at a flow rate of 10 ul/min over the activated and reference surfaces to allow for binding to chip-bound antigen. Next, binding buffer without HSA is sent over the chip at the same flow rate to allow for dissociation of bound HSA. After 10 min, remaining bound analyte is removed by injecting regeneration solution (50 mM NaOH).

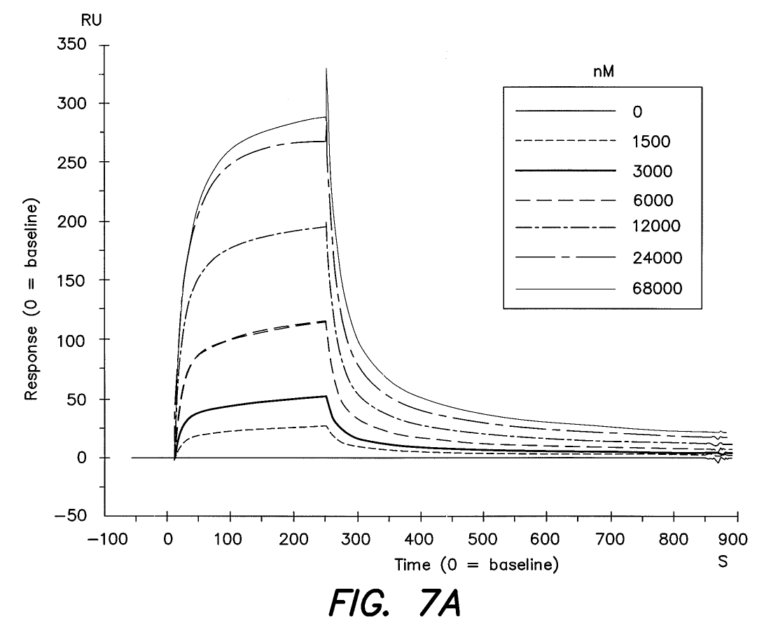

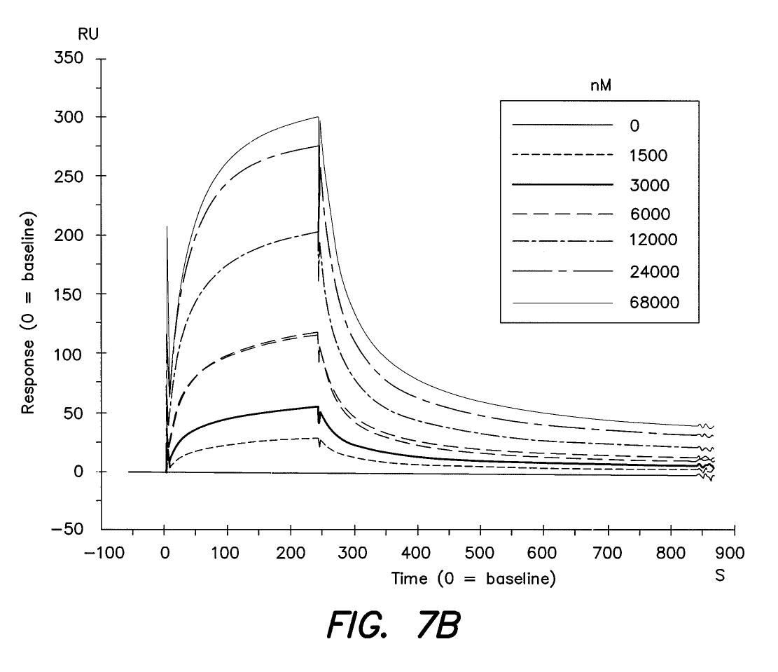

As depicted in FIGS. 7A and 7B, binding kinetics for both peptides were comparable. Though the dissociation curves are heterogeneous, dissociation rates between 2E-2 and 2E-3 1/s could be calculated. An increase in binding response for HSA concentrations higher than 15 μM is seen, but binding responses indicate that saturation levels is reached at 48 μM HSA. By fitting a 1:1 binding model, association rate constants were concentration dependent and range from 1E2-E4. These data indicate a binding affinity for the synthetic peptides described herein of at least more than 1 μM.

### Example 7

### Construction of a Nanobody-17D12 Fusion Protein and Analysis of Binding to HSA When Expressed on pIII of M13 Phage

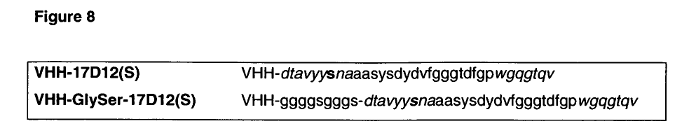

HSA-binding peptide 17D12 was genetically fused at the C-terminus of VHH, termed 2D3, that specifically binds to HER2 and is described in the US provisional application of Ablynx N.V. entitled “Amino acid sequences directed against HER2 and polypeptides comprising the same for the treatment of cancers and/or tumors” with a filing date of Nov. 27, 2007, see SEQ ID NO: 2060) with or without Gly₄Ser-Gly₃Ser linker and expressed on pIII of M13 phage (FIG. 8). The Cys residue in FR3 was replaced for a Ser.

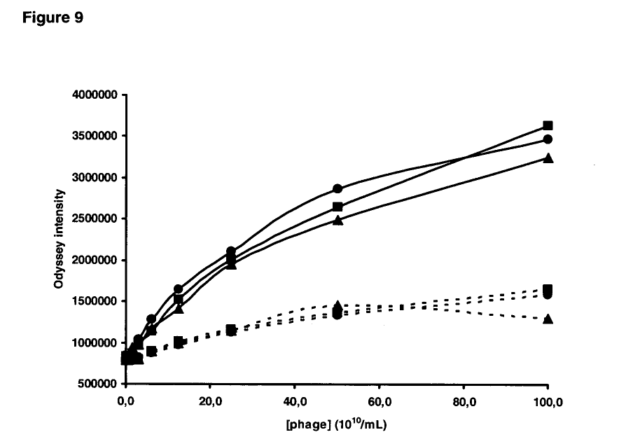

The phage binding assay to HSA and ovalbumin was performed essentially as described in example 4. Phages, produced in 10 mL culture volume, were incubated on both HSA and ovalbumin in a ½ dilution series, starting with 10¹² phages/mL. Dose-dependent binding to HSA was retained upon fusion of the peptide 17D12 at the C-terminus of a non-albumin binding intact VHH in the presence or absence of a GlySer linker (FIG. 9).

### Example 8

### Binding of 2D3-17D12 Fusion Protein to HSA

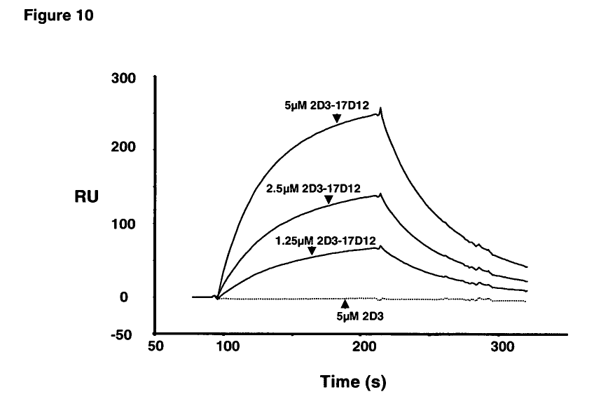

The 2D3-17D12 fusion protein EVQLVESGGSLVQPGGSLRLSCAASGFTFDDYAMSWVRQVPGKGLEWVSSINWSGT HTDYADSVKGRFTISRNNANNTLYLQMNSLKSEDTAVYYCAKNWRDAGTTWFEKS GSAGQGTQVTVSSDTAVYYCNAAASYSDYDVFGGGTDFGPWGQGTQVGGGS (SEQ ID NO: 30) was expressed in E. coli TG1 cells. The fusion protein was purified by IMAC/SEC and binding to HSA was assessed in BIAcore™ 3000. Therefore, a dilution series of 2D3-17D12 fusion protein and 2D3 Nanobody was injected on a CM5 chip coated with high density HSA (7000RU). FIG. 10 shows dose-dependent binding of 2D3-17D12 to HSA, whereas, as expected, 2D3 does not bind at identical concentrations tested. Calculated affinity of 2D3-17D12 for HSA is ˜10 μM. As a control, 2.5 μM 2D3-17D12 was injected on CM5 chip coated with high density of irrelevant protein (2400RU), but no specific binding was detected.

### Example 9

### Simultaneous Binding of 2D3-17D12 Fusion Protein to HER2 and HSA

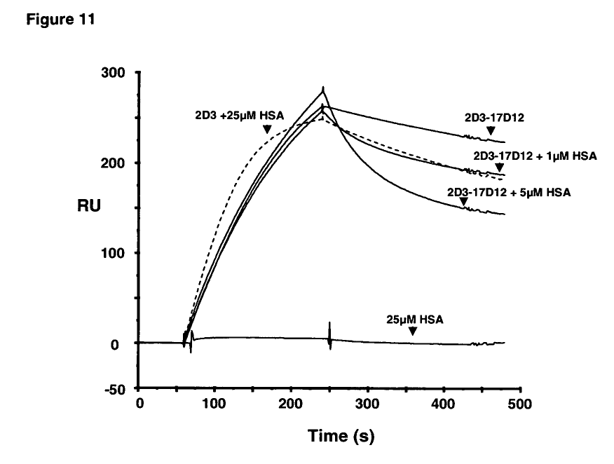

To examine whether the 2D3 Nanobody and the 17D12 CDR3 can simultaneously bind their respective target antigen (Her-2 antigen for 2D3 Nanobody, albumin for 17D12 CDR3), the following experiment was performed. 2D3-17D12 fusion protein was pre-incubated or not with increasing concentration of HSA and then injected on a CM5 chip coated with rhErbB2-Fc antigen (R&D Systems) at a density of ˜3000 RU. As shown in FIG. 11, injection of 2D3-17D12 premixed with HSA shows similar association rates compared to 2D3-17D12 alone with slightly higher but comparable off-rates compared to the control injection. The addition of HSA to 2D3 shows slightly different kinetics compared to 2D3-17D12 alone, but the off-rates are in similar range.

### Example 10

### Pharmacokinetic Analysis of Nanobody Genetically Fused to a CDR3 Loop with Binding Specificity to Albumin

By means of a non-limiting example, the half-life of compounds of the invention (such as the 2D3-17D12 fusion protein of SEQ ID NO:30) is determined by means of a pharmacokinetic study, performed in a rodent or non-human primate model, as follows. Groups of animals (n=2-10) are given an intravenous bolus injection of 1 mg/kg or 10 mg/kg 2D3-17D12 fusion protein. Plasma samples are obtained via a vein at different time points after dosing (e.g. 1, 2, 4, 6, 8, 12, 24, 48, 144, 192, 240, 288 and 336 h after dosing) and analyzed for the presence of the 2D3-17D12 fusion protein by ELISA. Plasma concentration versus time are fitted to a two-compartment elimination model. The pharmacokinetic parameters of clearance, VI, steady state volume (Vss), T½, AUC, and AUC corrected for actual dose administered (AUC/dose) are averaged for each treatment group. Differences between groups are determined by analysis of variance. Reference is also made to the references cited in the specification, as well as to Dennis et al., J. Biol. Chem 277:35035-42 (2002).

The sequences mentioned in the examples are listed in Table 2 below:

TABLE 2
sequences used in the Experimental Part

| | | |
|---|---|---|
| 17D12; | SEQ ID NO:1 | dtavyycnaaasysdy |
| | | dvfgggtdfgpwgqgt |
| | | qv |
| 17D12; | SEQ ID NO:2 | gacacggccgtttatt |
| | | attgtaatgcagccgc |
| | | ctcctatagcgactat |
| | | gacgtctttgggggag |
| | | gaactgactttggtcc |
| | | ctggggccaagggacc |
| | | caggtc |
| 17D2 CDR sequence; | SEQ ID NO:3: | aasysdydvfgggtdf |
| | | gp |
| primer ABL051; | SEQ ID NO:4 | ggctgagctgggtggt |
| | | cctgg |
| primer ABL0052; | SEQ ID NO:5 | ggctgagtttggtggt |
| | | cctgg |
| primer ABL003; | SEQ ID NO:6 | ggtacgtgctgttgaa |
| | | ctgttcc |
| primer For1Sfi; | SEQ ID NO:7 | Gtcctcgcaactgcgg |
| | | cccagccggccatggc |
| | | ggacacggccgbctat |
| | | tactg |
| primer For2Sfi; | SEQ ID NO:8 | Gtcctcgcaactgcgg |
| | | cccagccggccatggc |
| | | ggacacggccgtttat |
| | | wactg |
| primer For3Sfi; | SEQ ID NO:9 | Gtcctcgcaactgcgg |
| | | cccagccggccatggc |
| | | ggacacggccgtgtat |
| | | taytg |
| primer For4Sfi; | SEQ ID NO:10 | Gtcctcgcaactgcgg |
| | | cccagccggccatggc |
| | | ggacacggccgtctat |
| | | twttg |
| primer For5Sfi; | SEQ ID NO:11 | Gtcctcgcaactgcgg |
| | | cccagccggccatggc |
| | | ggacacggccgwttat |
| | | tattg |
| primer For6Sfi; | SEQ ID NO:12 | Gtcctcgcaactgcgg |
| | | cccagccggccatggc |
| | | ggacacggccatytat |
| | | twctg |
| primer For7Sfi; | SEQ ID NO:13 | Gtcctcgcaactgcgg |
| | | cccagccggccatggc |
| | | ggacacgggactytat |
| | | tactg |
| primer Back1Not; | SEQ ID NO:14 | gagtcattctcgactt |
| | | gcggccgctgaaccgc |
| | | ctccgacctgrgtbcc |
| | | ctggcccc |
| primer Back2Not; | SEQ ID NO:15 | gagtcattctcgactt |
| | | gcggccgctgaaccgc |
| | | ctccgacctkggtccc |
| | | ttkgcccc |
| primer Back3Not; | SEQ ID NO:16 | gagtcattctcgactt |
| | | gcggccgctgaaccgc |
| | | ctccgacctgggtccc |
| | | cggsccyc |
| primer Back4Not; | SEQ ID NO:17 | gagtcattctcgactt |
| | | gcggccgctgaaccgc |
| | | ctccgacctgggtccc |
| | | ctghcccc |
| primer Back5Not; | SEQ ID NO:18 | gagtcattctcgactt |
| | | gcggccgctgaaccgc |
| | | ctccgacctgggtccc |
| | | ctggccgt |
| primer Back1cysRNot; | SEQ ID NO:19 | gagtcattctcgactt |
| | | gcggccgctgaaccgg |
| | | ctccgacctgrgtbcc |
| | | ctggcacct |
| primer Back1cysWNot; | SEQ ID NO:20 | gagtcattctcgactt |
| | | gcggccgctgaaccgg |
| | | ctccgacctgrgtbcc |
| | | ctggcacca |
| primer Back2cysWNot; | SEQ ID NO:21 | gagtcattctcgactt |
| | | gcggccgctgaaccgg |
| | | ctccgacctkggtccc |
| | | ttkgcacca |
| primer Back3cysWNot; | SEQ ID NO:22 | gagtcattctcgactt |
| | | gcggccgctgaaccgg |
| | | ctccgacctgggtccc |
| | | cgggcacca |
| primer Back3cysRNot; | SEQ ID NO:23 | gagtcattctcgactt |
| | | gcggccgctgaaccgg |
| | | ctccgacctgggtccc |
| | | cgggcatct |
| primer Back4cysWNot; | SEQ ID NO:24 | gagtcattctcgactt |
| | | gcggccgctgaaccgg |
| | | ctccgacctgggtccc |
| | | ctggcacca |
| primer Back5cysWNot; | SEQ ID NO:25 | gagtcattctcgactt |
| | | gcggccgctgaaccgg |
| | | ctccgacctgggtccc |
| | | ctggcagta |
| 17D12-CDR3-NC; | SEQ ID NO:26 | aaasysdydvfgggtd |
| | | fgpa |
| 17D12-CDR3-C; | SEQ ID NO:27 | caaasysdydvfgggt |
| | | dfgpac |
| acetyl-AAASYSDYDVFGG | SEQ ID NO:28 | AAASYSDYDVFGGGTD |
| GTDFGP-c2 | | FGP |
| linker-biotin; | | |
| acetyl-CAAASYSDYDVFG | SEQ ID NO:29; | CAAASYSDYDVFGGGT |
| GGTDFGP-c2 | | DFGP |
| linker-biotin, | | |
| 2D3-17D12 fusion | SEQ ID NO:30 | EVQLVESGGSLVQPGG |
| protein; | | SLRLSCAASGFTFDDY |
| | | AMSWVRQVPGKGLEWV |
| | | SSINWSGTHTDYADSV |
| | | KGRFTISRNNANNTLY |
| | | LQMNSLKSEDTAVYYC |
| | | AKNWRDAGTTWFEKSG |
| | | SAGQGTQVTVSSDTAV |
| | | YYCNAAASYSDYDVFG |
| | | GGTDFGPWGQGTQVGG |
| | | GS |

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

1. Amino acid sequence that can bind to a serum protein and that essentially consists of a CDR sequence.

2. Amino acid sequence that can bind to a serum protein and that comprises a CDR sequence (and in particular, a single CDR sequence), wherein said amino acid sequence does not comprise an immunoglobulin fold and/or is not capable of forming an immunoglobulin fold.

3. Amino acid sequence according to claim 2, in which said CDR sequence can bind to a serum protein.

4. acid sequence according to claim 2, in which said CDR sequence is derived from an immunoglobulin variable domain that can bind to a serum protein; and/or in which said amino acid sequence essentially consists of a fragment of an immunoglobulin variable domain that comprises a CDR sequence.

5. Amino acid sequence according to claim 2, in which said CDR sequence is derived from an immunoglobulin variable domain, which is selected from the group consisting of a V_{H}-domain, a V_{L}-domain, a V_{HH}-domain or an antigen-binding fragment of an immunoglobulin variable domain; and/or is a fragment of a V_{H}-domain, a V_{L}-domain, a V_{HH}-domain or an antigen-binding fragment of an immunoglobulin variable domain that comprises a CDR sequence.

6. Amino acid sequence according to claim 2, in which said CDR sequence is derived from an immunoglobulin variable domain, which is selected from the group consisting of a human variable domain, a (single) domain antibody, a dAb, or a Nanobody®; and/or is a fragment of a human variable domain, a (single) domain antibody, a dAb, or a Nanobody®.

7. Amino acid sequence according to claim 2, in which said CDR sequence is a CDR2 sequence.

8. Amino acid sequence according to claim 2, in which said CDR sequence is a CDR3 sequence.

9. Amino acid sequence according to claim 2, in which said CDR sequence has a length between 3 and 40 amino acid residues, preferably between 5 and 30 amino acid residues.

10. Amino acid sequence according to claim 2, in which said amino acid sequence binds to a serum protein in such a way that the half-life of the serum protein molecule is not (significantly) reduced.

11. Amino acid sequence according to claim 2, in which said amino acid sequence binds to a serum protein chosen from the group consisting of serum albumin, serum immunoglobulins such as IgG, thyroxine-binding protein, transferrin, fibrinogen; or to at least one part, fragment, epitope or domain of any of the foregoing.

12. Amino acid sequence according to claim 2, in which said amino acid sequence binds to serum albumin or at least one part, fragment, epitope or domain thereof.

13. Amino acid sequence according to claim 2, in which said amino acid sequence binds to human serum albumin or at least one part, fragment, epitope or domain thereof.

14. Amino acid sequence according to claim 12, which is capable of binding to amino acid residues on serum albumin that are not involved in binding of (human) serum albumin to FcRn.

15. Amino acid sequence according to claim 12, which is capable of binding to amino acid residues on serum albumin that do not form part of domain III of human serum albumin.

16. Amino acid sequence according to claim 1, in which the CDR sequence is flanked by two flanking amino acid sequences on either side of the CDR sequence.

17. Amino acid sequence according to claim 16, in which said two flanking amino acid sequences each have a length of between 1 and 30 amino acid residues, preferably between 2 and 20 amino acid residues, such as about 5, 10 or 15 amino acid residues.

18. Amino acid sequence according to claim 16, in which said two flanking amino acid sequences are derived from immunoglobulin framework sequences; and/or are fragments of immunoglobulin framework sequences.

19. Amino acid sequence according to claim 18, in which said CDR sequence is derived from a CDR sequence from an immunoglobulin variable domain and in which said two flanking amino acid sequences are immunoglobulin framework sequences that have been derived from the framework sequences that, in the immunoglobulin variable domain from which said CDR sequence is derived, are adjacent to said CDR sequence; and/or are fragments of the framework sequences that, in the immunoglobulin variable domain from which said CDR sequence is derived, are adjacent to said CDR sequence.

20. Amino acid sequence according to claim 19, in which said CDR sequence is a CDR2 sequence and in which said flanking sequences are immunoglobulin framework sequences that have been derived from a framework 2 sequence and a framework 3 sequence, respectively; and/or are fragments of a framework 2 sequence and a framework 3 sequence, respectively.

21. Amino acid sequence according to claim 19, in which said CDR sequence is a CDR3 sequence and in which said flanking sequences are immunoglobulin framework sequences that have been derived from a framework 3 sequence and a framework 4 sequence, respectively; and/or are fragments of a framework 3 sequence and a framework 3 sequence, respectively.

22. Amino acid sequence according to claim 1 that contains at least two cysteine residues that are capable of forming a disulphide bridge.

23. Amino acid sequence according to claim 22, in which the CDR sequence is flanked by two flanking amino acid sequences on either side of the CDR sequence, and in which each flanking amino acid sequence contains at least one cysteine residue that is capable of forming a disulphide bridge.

24. Amino acid sequence according to claim 22, in which said two flanking amino acid sequences are derived from immunoglobulin framework sequences, and in which the at least two cysteine residues that are capable of forming a disulphide bridge are either cysteine residues that naturally occur in said immunoglobulin framework sequences and/or are cysteine residues that have been introduced into said in immunoglobulin framework sequences.

25. Amino acid sequence according to claim 1, that comprises at least one disulfide bridge.

26. Compound or construct which comprises at least one amino acid sequence according to claim 1 and at least one therapeutic moiety.

27. Compound or construct which comprises at least one amino acid sequence according to claim 22 and at least one therapeutic moiety.

28. Compound or construct which comprises at least one amino acid sequence according to claim 25 and at least one therapeutic moiety.

29. Compound or construct according to claim 27, in which the at least one amino acid sequence is either directly linked to the at least one therapeutic moiety or is linked to the at least one therapeutic moiety via one or more suitable linkers or spacers.

30. Compound or construct according to claim 26, in which the at least one therapeutic moiety comprises or essentially consists of an amino acid sequence.

31. Compound or construct according to claim 26, in which the at least one therapeutic moiety comprises or essentially consists of an immunoglobulin sequence or an antigen-binding fragment thereof, such as an immunoglobulin variable domain or an antigen-binding fragment thereof; or a protein or polypeptide comprising the same.

32. Compound or construct according to claim 31, in which said therapeutic moiety comprises or essentially consists of a (single) domain antibody, a “dAb”, or a Nanobody®.

33. Compound or construct according to claim 29, in which the at least one amino acid sequence is either directly linked to the at least one therapeutic moiety or is linked to the at least one therapeutic moiety via at one or more suitable linkers or spacers, in which said at least linkers or spacers comprise or essentially consist of amino acid sequences.

34. Compound or construct according to claim 29, which comprises or essentially consist of a (fusion) protein or (fusion) polypeptide, comprising the at least one amino acid sequence and the at least one therapeutic moiety.

35. Compound or construct, which comprises or essentially consist of a (fusion) protein or (fusion) polypeptide, comprising at least one amino acid sequence according to claim 24 and at least one therapeutic moiety.

36. Compound or construct, which comprises or essentially consist of a (fusion) protein or (fusion) polypeptide, comprising at least one amino acid sequence according to claim 25 and the at least one therapeutic moiety.

37. Nucleotide sequence or nucleic acid that encodes an amino acid sequence according to claim 1.

38. Nucleotide sequence or nucleic acid that encodes an amino acid sequence according to claim 22.

39. Host or host cell that contains a nucleotide sequence or nucleic acid according to claim 37.

40. Host or host cell that contains a nucleotide sequence or nucleic acid according to claim 38.

41. Method for preparing an amino acid sequence, said method comprising at least the step of forming a disulphide bridge in an amino acid sequence according to claim 22.

42. Method for preparing a compound or construct, said method comprising at least the step of forming a disulphide bridge in an compound or construct according to claim 27, in the part of said compound or construct that corresponds to the amino acid sequence.

43. Method for preparing a compound or construct, said method comprising at least the step of forming a disulphide bridge in an compound or construct according to claim 34, in the part of said compound or construct that corresponds to the amino acid sequence.

44. Method for preparing an amino acid sequence, which method at least comprises the step of:
a) expressing a nucleotide sequence or nucleic acid of according to claim 37;
and optionally further comprises:
b) isolating the amino acid sequence encoded by the nucleotide sequence or nucleic acid so expressed.

45. Method for preparing an amino acid sequence, said method at least comprising:
a) cultivating or maintaining a host or host cell according to claim 39 under conditions such that said host or host cell produces an amino acid sequence encoded by the nucleotide sequence or nucleic acid;
and optionally further comprising:
b) isolating the amino acid sequence obtained in step a).

46. Method for preparing an amino acid sequence, which method at least comprises the steps of
a) expressing a nucleotide sequence or nucleic acid according to claim 38, and
b) optionally further comprising isolating the amino acid sequence encoded by the nucleotide sequence or nucleic acid so expressed; and
c) forming a disulphide bridge in the amino acid sequence.

47. Method for preparing an amino acid sequence, said method at least comprising the steps of:
a) cultivating or maintaining a host or host cell according to claim 40 under conditions such that said host or host cell produces an amino acid sequence encoded by the nucleotide sequence or nucleic acid and
b) optionally further comprising isolating the amino acid sequence so produced; and
c) forming a disulphide bridge in the amino acid sequence.

48. Amino acid sequence, compound or construct, obtained via the method of claim 41.

49. Pharmaceutical composition that comprises at least one amino acid sequence according to claim 1; and optionally at least one pharmaceutically acceptable carrier, diluent or excipient.

50. Method for generating an amino acid sequence according to claim 1, which method at least comprises the steps of:
a) providing a set, collection or library of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold;
b) screening said set, collection or library for amino acid sequences that can bind to and/or have affinity for a serum protein or at least one part, fragment, epitope or domain thereof; and
c) isolating the amino acid sequence(s) that can bind to and/or have affinity for said serum protein or said at least one part, fragment, epitope or domain thereof.

51. Method according to claim 50, in which, in step b), said set, collection or library of amino acid sequences is screened for amino acid sequences that can bind to and/or have affinity for a serum protein chosen from the group consisting of serum albumin, serum immunoglobulins such as IgG, thyroxine-binding protein, transferrin or fibrinogen; and/or for amino acid sequences that can bind to and/or have affinity for at least one part, fragment, epitope or domain of serum albumin, serum immunoglobulins such as IgG, thyroxine-binding protein, transferrin or fibrinogen.

52. Method according to claim 51, in which, in step b), said set, collection or library of amino acid sequences is screened for amino acid sequences that can bind to and/or have affinity for serum albumin or at least one part, fragment, epitope or domain thereof.

53. Method according to claim 52, in which, in step b), said set, collection or library of amino acid sequences is screened for amino acid sequences that can bind to and/or have affinity for human serum albumin or at least one part, fragment, epitope or domain thereof.

54. Method according to claim 53, in which, in step b), said set, collection or library of amino acid sequences is screened for one or more amino acid sequences that can bind to and/or have affinity for a part, fragment, epitope or domain of human serum albumin that is not involved in binding of human serum albumin to FcRn.

55. Method according to claim 53, in which, in step b), said set, collection or library of amino acid sequences is screened for amino acid sequences that can bind to and/or have affinity for at least one part, fragment, epitope or domain of human serum albumin that does not form part of domain III of human serum albumin.

56. Method according to claim 50, in which, during step b), the set, collection or library of amino acid sequences is displayed on a phage, phagemid, ribosome or suitable micro-organism.

57. Method according to claim 50, in which the set, collection or library of amino acid sequences used in step a) comprises a set, collection or library of amino acid sequences that essentially consist of a CDR sequence flanked by two flanking amino acid sequences that have been derived from the immunoglobulin framework sequences; and/or of fragments of immunoglobulin sequences that comprise a CDR sequence flanked on both sides by framework sequences or fragments of framework sequences.

58. Method according to claim 57, in which the set, collection or library of amino acid sequences used in step a) comprises a set, collection or library of amino acid sequences that comprise or essentially consist of a CDR sequence flanked by two flanking amino acid sequences that have been derived from the framework sequences that, in the immunoglobulin variable domain from which said CDR sequence is derived, are adjacent to said CDR sequence.

59. Method according to claim 58, in which the set, collection or library of amino acid sequences used in step a) comprises a set, collection or library of amino acid sequences that comprise or essentially consist of a CDR2 sequence flanked by two flanking amino acid sequences that have been derived from a framework 2 sequence and a framework 3 sequence, respectively.

60. Method according to claim 58, in which the set, collection or library of amino acid sequences used in step a) comprises a set, collection or library of amino acid sequences that comprise or essentially consist of a CDR3 sequence flanked by two flanking amino acid sequences that have been derived from a framework 3 sequence and a framework 4 sequence, respectively.

61. Method according to claim 57, which optionally further comprises introducing (i.e. by adding, inserting or substituting) of one or two cysteine residues, such that each framework sequence in the resulting amino acid sequence contains at least one cysteine residue.

62. Method according to claim 50, wherein the set, collection or library of amino acid sequences used in step a) has been obtained by a method that at least comprises the steps of
a) providing a set, collection or library of nucleotide sequences that encode immunoglobulin sequences;
b) amplifying said nucleotide sequences using a combination of site-specific primers, such that the amplified fragments encode a set, library or collection of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold; and
c) expressing the amplified fragments obtained in step b), so as to provide a set, library or collection of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold.

63. Method according to claim 62, in which the set, collection or library of nucleotide sequences that encode immunoglobulin sequences used in step a) is an immune set, collection or library.

64. Method according to claim 63, in which the set, collection or library of nucleotide sequences that encode immunoglobulin sequences used in step a) is an immune set, collection or library that has been obtained from mammal that has been suitably immunized with a serum protein (i.e. so as to raise an immune response against said serum protein).

65. Method according to claim 64, in which the set, collection or library of nucleotide sequences that encode immunoglobulin sequences used in step a) is an immune set, collection or library of nucleotide sequences that encode heavy chain antibodies or V_{HH} sequences, that have been obtained from a Camelid that has been suitably immunized with serum protein (i.e. so as to raise an immune response against said serum protein).

66. Method according to claim 62, in which said site-specific primers are specific for and/or capable of hybridizing to (i.e. under the conditions used for the amplification) nucleotide sequences that encode the framework sequences that flank said CDR sequence.

67. Method according to claim 62, in which, in step b), said nucleotide sequences are amplified using a combination of site-specific primers, such that the amplified fragments encode a set, library or collection of amino acid sequences that (i) essentially consist of a CDR2 sequence; and/or (ii) comprise a fragment of an immunoglobulin that comprises a CDR2 sequences; and/or (iii) comprise a CDR2 sequence but that do not comprise an immunoglobulin fold and are also not capable of forming an immunoglobulin fold.

68. Method according to claim 66, in which said site-specific primers are specific for and/or capable of hybridizing to (i.e. under the conditions used for the amplification) nucleotide sequences that encode framework 2 sequences and framework 3 sequences, respectively.

69. Method according to claim 62, in which, in step b), said nucleotide sequences are amplified using a combination of site-specific primers, such that the amplified fragments encode a set, library or collection of amino acid sequences that (i) essentially consist of a CDR3 sequence; and/or (ii) comprise a fragment of an immunoglobulin that comprises a CDR3 sequences; and/or (iii) comprise a CDR3 sequence but that do not comprise an immunoglobulin fold and are also not capable of forming an immunoglobulin fold.

70. Method according to claim 68, in which said site-specific primers are specific for and/or capable of hybridizing to (i.e. under the conditions used for the amplification) nucleotide sequences that encode framework 3 sequences and framework 4 sequences, respectively.

71. Method according to claim 50, wherein the set, collection or library of amino acid sequences used in step a) has been obtained by a method that at least comprises a step of affinity maturation.

72. Method for generating an amino acid sequence according to claim 1, which method at least comprises the steps of:
a) providing a set, collection or library of immunoglobulin sequences;
b) screening said set, collection or library of immunoglobulin sequences for immunoglobulin sequences that can bind to and/or have affinity for a serum protein or at least one part, fragment, epitope or domain thereof;
c) determining the nucleotide sequence and/or the amino acid sequence of at least one immunoglobulin sequence that can bind to and/or has affinity for a serum protein or at least one part, fragment, epitope or domain thereof, as identified during step b); and/or determining the nucleotide sequence and/or the amino acid sequence of a CDR sequence thereof and/or of a fragment thereof that comprises a CDR sequence; and
d) preparing, using any suitable technique known per se, an amino acid sequence according to claim 1 that (i) essentially consist of a CDR sequence with an amino acid sequence that has been determined in step c); and/or (ii) comprises a fragment of an immunoglobulin with an amino acid sequence that has been determined in step c); and/or (iii) comprises a CDR sequence with an amino acid sequence that has been determined in step c), but that does not comprise an immunoglobulin fold and are also not capable of forming an immunoglobulin fold.

73. Method according to claim 62, in which, in step b), said set, collection or library of immunoglobulin sequences is screened for immunoglobulin sequences that can bind to and/or have affinity for a serum protein chosen from the group consisting of serum albumin, serum immunoglobulins such as IgG, thyroxine-binding protein, transferrin or fibrinogen; and/or for immunoglobulin sequences that can bind to and/or have affinity for at least one part, fragment, epitope or domain of serum albumin, serum immunoglobulins such as IgG, thyroxine-binding protein, transferrin or fibrinogen.

74. Method according to claim 63, in which, in step b), said set, collection or library of immunoglobulin sequences is screened for immunoglobulin sequences that can bind to and/or have affinity for serum albumin or at least one part, fragment, epitope or domain thereof.

75. Method according to claim 74, in which, in step b), said set, collection or library of immunoglobulin sequences is screened for immunoglobulin sequences that can bind to and/or have affinity for human serum albumin or at least one part, fragment, epitope or domain thereof.

76. Method according to claim 75, in which, in step b), said set, collection or library of immunoglobulin sequences is screened for one or more immunoglobulin sequences that can bind to and/or have affinity for a part, fragment, epitope or domain of (human) serum albumin that is not involved in binding of (human) serum albumin to FcRn.

77. Method according to claim 75, in which, in step b), said set, collection or library of immunoglobulin sequences is screened for immunoglobulin sequences that can bind to and/or have affinity for at least one part, fragment, epitope or domain of (human) serum albumin that does not form part of domain III of (human) serum albumin.

78. Method according to claim 72, in which, during step b), the set, collection or library of immunoglobulin sequences is displayed on a phage, phagemid, ribosome or suitable micro-organism.

79. Method according to claim 72, wherein the set, collection or library of immunoglobulin sequences is a naïve set, collection or library of immunoglobulin sequences.

80. Method according to claim 72, wherein the set, collection or library of immunoglobulin sequences is a synthetic or semi-synthetic set, collection or library of immunoglobulin sequences.

81. Method according to claim 72, wherein the set, collection or library of immunoglobulin sequences is a set, collection or library of immunoglobulin sequences that have been subjected to affinity maturation.

82. Method according to claim 72, wherein the set, collection or library of immunoglobulin sequences is an immune set, collection or library of immunoglobulin sequences.

83. Method according to claim 82, in which the set, collection or library of nucleotide sequences that encode immunoglobulin sequences used in step a) is an immune set, collection or library that has been obtained from mammal that has been suitably immunized with a serum protein (i.e. so as to raise an immune response against said serum protein).

84. Method according to claim 83, in which the set, collection or library of immunoglobulin sequences used in step a) is an immune set, collection or library of heavy chain antibodies or V_{HH} sequences, that have been obtained from a Camelid that has been suitably immunized with serum protein (i.e. so as to raise an immune response against said serum protein).

85. Method according to claim 72, wherein the set, collection or library of immunoglobulin sequences is a set, collection or library of CDR sequences derived from heavy chain variable domains or of light chain variable domains.

86. Method according to claim 85, wherein the set, collection or library of immunoglobulin sequences is a set, collection or library of domain antibodies, single domain antibodies or immunoglobulin sequences that are capable of functioning as a domain antibody or single domain antibody.

87. Method according to claim 72, wherein said CDR sequence is a CDR2 sequence.

88. Method according to claim 72, wherein said CDR sequence is a CDR3 sequence.

89. Method for generating an amino acid sequence according to claim 1, which method at least comprises the steps of:
a) providing a set, collection or library of cells, derived from a Camelid, that express immunoglobulin sequences;
b) screening said set, collection or library of cells for (i) cells that express immunoglobulin sequences that can bind to and/or have affinity for a serum protein or at least one part, fragment, epitope or domain thereof; and (ii) cells that express heavy chain antibodies; in which substeps (i) and (ii) can be performed essentially as a single screening step or in any suitable order as two separate screening steps, so as to provide at least one cell that expresses heavy chain antibody that can bind to and/or has affinity for at least one domain or epitope of a serum protein;
c) determining the nucleotide sequence and/or the amino acid sequence of at least one heavy chain antibody, expressed by a cell provided in step b), that can bind to and/or has affinity for a serum protein or at least one part, fragment, epitope or domain thereof; and/or determining the nucleotide sequence and/or the amino acid sequence of a CDR sequence thereof and/or of a fragment thereof that comprises a CDR sequence; and
d) preparing, using any suitable technique known per se, an amino acid sequence according to claim 1 that (i) essentially consist of a CDR sequence with an amino acid sequence that has been determined in step c); and/or (ii) comprises a fragment of an immunoglobulin with an amino acid sequence that has been determined in step c); and/or (iii) comprises a CDR sequence with an amino acid sequence that has been determined in step c), but that does not comprise an immunoglobulin fold and are also not capable of forming an immunoglobulin fold.

90. Method according to claim 89, wherein the collection or sample of cells is a collection or sample of B-cells.

91. Method according to claim 89, wherein the collection or sample of cells is obtained from a Camelid that has been suitably immunized with an antigen that comprises the desired domain or epitope(s) of a serum protein, such that an immune response against the desired domain or epitope(s) is raised.

92. Method according to claim 89, wherein the screening of step b) is performed using a flow cytometry technique such as FACS.

93. Method for generating a nucleotide sequence that encodes an amino acid sequence according to claim 1, which method at least comprises the steps of:
a) providing a set, collection or library of nucleotide sequences that encode amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold;
b) screening said set, collection or library for nucleotide sequences that encode amino acid sequences that can bind to and/or have affinity for a serum protein or at least one part, fragment, epitope or domain thereof; and
c) isolating the nucleotide sequence(s) that encode amino acid sequence(s) that can bind to and/or have affinity for said serum protein or said at least one part, fragment, epitope or domain thereof.

94. Method according to claim 93, in which, in step b), said set, collection or library of nucleotide sequences is screened for nucleotide sequences that encode amino acid sequences that can bind to and/or have affinity for a serum protein chosen from the group consisting of serum albumin, serum immunoglobulins such as IgG, thyroxine-binding protein, transferrin or fibrinogen; and/or for amino acid sequences that can bind to and/or have affinity for at least one part, fragment, epitope or domain of serum albumin, serum immunoglobulins such as IgG, thyroxine-binding protein, transferrin or fibrinogen.

95. Method according to claim 94, in which, in step b), said set, collection or library of nucleotide sequences is screened for nucleotide sequences that encode amino acid sequences that can bind to and/or have affinity for serum albumin or at least one part, fragment, epitope or domain thereof.

96. Method according to claim 95, in which, in step b), said set, collection or library of nucleotide sequences is screened for nucleotide sequences that encode amino acid sequences that can bind to and/or have affinity for human serum albumin or at least one part, fragment, epitope or domain thereof.

97. Method according to claim 96, in which, in step b), said set, collection or library of nucleotide sequences is screened for one or more nucleotide sequences that encode amino acid sequences that can bind to and/or have affinity for a part, fragment, epitope or domain of (human) serum albumin that is not involved in binding of (human) serum albumin to FcRn.

98. Method according to claim 96, in which, in step b), said set, collection or library of nucleotide sequences is screened for nucleotide sequences that encode amino acid sequences that can bind to and/or have affinity for at least one part, fragment, epitope or domain of (human) serum albumin that does not form part of domain III of (human) serum albumin.

99. Method according to claim 93, in which, during step b), the set, collection or library of nucleotide sequences is displayed as amino acid sequences on a phage, phagemid, ribosome or suitable micro-organism.

100. Method according to claim 93, in which the set, collection or library of nucleotide sequences used in step a) comprises a set, collection or library of nucleotide sequences that encode amino acid sequences that essentially consist of a CDR sequence flanked by two flanking amino acid sequences that have been derived from the immunoglobulin framework sequences; and/or of fragments of immunoglobulin sequences that comprise a CDR sequence flanked on both sides by framework sequences or fragments of framework sequences.

101. Method according to claim 100, in which the set, collection or library of nucleotide sequences used in step a) comprises a set, collection or library of nucleotide sequences that encode amino acid sequences that comprise or essentially consist of a CDR sequence flanked by two flanking amino acid sequences that have been derived from the framework sequences that, in the immunoglobulin variable domain from which said CDR sequence is derived, are adjacent to said CDR sequence.

102. Method according to claim 101, in which the set, collection or library of nucleotide sequences used in step a) comprises a set, collection or library of nucleotide sequences that encode amino acid sequences that comprise or essentially consist of a CDR2 sequence flanked by two flanking amino acid sequences that have been derived from a framework 2 sequence and a framework 3 sequence, respectively.

103. Method according to claim 102, in which the set, collection or library of nucleotide sequences used in step a) comprises a set, collection or library of nucleotide sequences that encode amino acid sequences that comprise or essentially consist of a CDR3 sequence flanked by two flanking amino acid sequences that have been derived from a framework 3 sequence and a framework 4 sequence, respectively.

104. Method according to claim 100, which optionally further comprises introducing (i.e. by adding, inserting or substituting one or more nucleotides) codons that encode one or two cysteine residues, such that each framework sequences in the amino acid sequence that is encoded by the nucleotide sequence thus obtained encodes contains at least one cysteine residue.

105. Method according to claim 93, wherein the set, collection or library of nucleotide sequences used in step a) has been obtained by a method that at least comprises the steps of
a) providing a set, collection or library of nucleotide sequences that encode immunoglobulin sequences;
b) amplifying said nucleotide sequences using a combination of site-specific primers, such that the amplified fragments encode a set, library or collection of amino acid sequences that (i) essentially consist of a CDR sequence; and/or (ii) comprise a fragment of an immunoglobulin that comprises a CDR sequences; and/or (iii) comprise a CDR sequence but that do not comprise an immunoglobulin fold and are also not capable of forming an immunoglobulin fold.

106. Method according to claim 105, in which the set, collection or library of nucleotide sequences used in step a) is an immune set, collection or library.

107. Method according to claim 105, in which the set, collection or library of nucleotide sequences that encode immunoglobulin sequences used in step a) is an immune set, collection or library that has been obtained from mammal that has been suitably immunized with a serum protein (i.e. so as to raise an immune response against said serum protein).

108. Method according to claim 107, in which the set, collection or library of nucleotide sequences that encode immunoglobulin sequences used in step a) is an immune set, collection or library of nucleotide sequences that encode heavy chain antibodies or VHH sequences, that have been obtained from a Camelid that has been suitably immunized with serum protein (i.e. so as to raise an immune response against said serum protein).

109. Method according to claim 105, in which said site-specific primers are specific for and/or capable of hybridizing to (i.e. under the conditions used for the amplification) nucleotide sequences that encode the framework sequences that flank said CDR sequence.

110. Method according to claim 105, in which, in step b), said nucleotide sequences are amplified using a combination of site-specific primers, such that the amplified fragments encode a set, library or collection of amino acid sequences that (i) essentially consist of a CDR2 sequence; and/or (ii) comprise a fragment of an immunoglobulin that comprises a CDR2 sequences; and/or (iii) comprise a CDR2 sequence but that do not comprise an immunoglobulin fold and are also not capable of forming an immunoglobulin fold.

111. Method according to claim 109, in which said site-specific primers are specific for and/or capable of hybridizing to (i.e. under the conditions used for the amplification) nucleotide sequences that encode framework 2 sequences and framework 3 sequences, respectively.

112. Method according to claim 105, in which, in step b), said nucleotide sequences are amplified using a combination of site-specific primers, such that the amplified fragments encode a set, library or collection of amino acid sequences that (i) essentially consist of a CDR3 sequence; and/or (ii) comprise a fragment of an immunoglobulin that comprises a CDR3 sequences; and/or (iii) comprise a CDR3 sequence but that do not comprise an immunoglobulin fold and are also not capable of forming an immunoglobulin fold.

113. Method according to claim 111, in which said site-specific primers are specific for and/or capable of hybridizing to (i.e. under the conditions used for the amplification) nucleotide sequences that encode framework 3 sequences and framework 4 sequences, respectively.

114. Method according to claim 50, wherein the set, collection or library of nucleotide sequences used in step a) encodes amino acid sequences that have been obtained by a method that at least comprises a step of affinity maturation.

115. Method according to claim 93, which further comprises the step of expressing the nucleotide sequence thus obtained.

116. Method according to claim 93, which further comprises the step(s) of linking one or more of the nucleotide sequence thus obtained to each other and/or to one or more nucleotide sequences that encode a therapeutic moiety that comprises or essentially consists of an amino acid sequence, optionally via one or more nucleotide sequence that encode one or more linkers, so as to provide a nucleotide sequence that encodes an amino acid sequence that can bind to a serum protein and that essentially consists of a CDR sequence.

117. Amino acid sequence that can bind to a serum protein and that comprises at least one disulfide bridge.

118. Amino acid sequence according to claim 117, which has a length of less than 90 amino acid residues, preferably less than 50 amino acid residues, such as about 40, 30 or 20 amino acid residues.

119. Amino acid sequence according to claim 117, comprising or essentially consisting of a peptide sequence that can bind to a serum protein flanked by two flanking amino acid sequences, in which each flanking amino acid sequence contains a cysteine residue that forms part of the disulfide bridge.

120. Amino acid sequence according to claim 119, in which said peptide sequence has a length between 3 and 30 amino acid residues, preferably between 5 and 25 amino acid residues.

121. Amino acid sequence according to claim 119, in which said two flanking amino acid sequences each have a length of between 1 and 30 amino acid residues, preferably between 2 and 20 amino acid residues, such as about 5, 10 or 15 amino acid residues.

122. Amino acid sequence according to claim 119, in which said two flanking amino acid sequences are derived from immunoglobulin framework sequences and/or are fragments of immunoglobulin framework sequences.

123. Amino acid sequence according to claim 122, in which said two flanking amino acid sequences are derived from immunoglobulin framework sequences, and in which the cysteine residue in each flanking amino acid sequence that forms part of the disulphide bridge is either a cysteine residue that naturally occurs in said immunoglobulin framework sequences (or in said fragment thereof) and/or is a cysteine residue that has been introduced into said in immunoglobulin framework sequence (or in said fragment thereof).

124. Amino acid sequence according to claim 119, in which said peptide sequence is a synthetic peptide sequence.

125. Amino acid sequence according to claim 119, in which said peptide sequence is a sequence that has been generated using an affinity maturation technique.

126. Amino acid sequence according to claim 119, in which said peptide sequence essentially consists of a CDR sequence.

127. Amino acid sequence according to claim 126, in which said peptide sequence essentially consists of a CDR sequence that has been derived from an V_{H}-, V_{L}- or V_{HH}-sequence that can bind to a serum protein.

128. Amino acid sequence according to claim 126, in which said peptide sequence essentially consists of a CDR sequence that has been derived from a (single) domain antibody, a dAb, or a Nanobody® or a fragment thereof.

129. Amino acid sequence according to claim 126, in which said peptide sequence essentially consists of a CDR2 sequence

130. Amino acid sequence according to claim 129, in which one of the two flanking amino acid sequences is derived from a framework 2 sequence and/or a fragment of a framework 2 sequence, and in which the other flanking amino acid sequence is derived from a framework 3 sequence and/or is a fragment of a framework 3 sequence, respectively.

131. Amino acid sequence according to claim 126, in which said peptide sequence essentially consists of a CDR3 sequence.

132. Amino acid sequence according to claim 130, in which one of the two flanking amino acid sequences is derived from a framework 3 sequence and/or a fragment of a framework 3 sequence, and in which the other flanking amino acid sequence is derived from a framework 4 sequence and/or is a fragment of a framework 4 sequence, respectively.

133. Amino acid sequence according to claim 117, which can bind to a serum protein in such a way that the half-life of the serum protein molecule is not (significantly) reduced.

134. Amino acid sequence according to claim 117, which can bind to a serum protein from the group consisting of serum albumin, serum immunoglobulins, thyroxine-binding protein, transferrin, fibrinogen or fragments thereof.

135. Amino acid sequence according to claim 117, which can bind to a serum albumin or a fragment thereof.

136. Amino acid sequence according to claim 135, which can bind to human serum albumin or a fragment thereof.

137. Amino acid sequence according to claim 136, which is capable of binding to amino acid residues on (human) serum albumin that are not involved in binding of serum albumin to FcRn.

138. Amino acid sequence according to claim 134, which is capable of binding to amino acid residues on (human) serum albumin that do not form part of domain III of serum albumin.

139. Compound or construct which comprises at least one amino acid sequence according to claim 117 and at least one therapeutic moiety.

140. Compound or construct according to claim 139, in which the at least one amino acid sequence is either directly linked to the at least one therapeutic moiety or is linked to the at least one therapeutic moiety via one or more suitable linkers or spacers.

141. Compound or construct according to claim 139, in which the at least one therapeutic moiety comprises or essentially consists of an amino acid sequence.

142. Compound or construct according to claim 139, in which the at least one therapeutic moiety comprises or essentially consists of an immunoglobulin sequence or an antigen-binding fragment thereof, such as an immunoglobulin variable domain or an antigen-binding fragment thereof; or a protein or polypeptide comprising the same.

143. Compound or construct according to claim 142, in which said therapeutic moiety comprises or essentially consists of a (single) domain antibody, a “dAb”, or a Nanobody®.

144. Compound or construct according to claim 141, in which the at least one amino acid sequence is either directly linked to the at least one therapeutic moiety or is linked to the at least one therapeutic moiety via at one or more suitable linkers or spacers, in which said at least linkers or spacers comprise or essentially consist of amino acid sequences.

145. Compound or construct according to claim 139, which comprises or essentially consist of a (fusion) protein or (fusion) polypeptide, comprising the at least one amino acid sequence and the at least one therapeutic moiety.

146. Compound or construct, which comprises or essentially consist of a (fusion) protein or (fusion) polypeptide, comprising at least one amino acid sequence according to claim 119 and at least one therapeutic moiety that comprises or essentially consists of an amino acid sequence.

147. Nucleotide sequence or nucleic acid that encodes an amino acid sequence with the same primary amino acid sequence as an amino acid sequence according to claim 117.

148. Host or host cell that contains a nucleotide sequence or nucleic acid according to claim 147.

149. Method for preparing an amino acid sequence, said method comprising at least the steps of:
a) providing an amino acid sequence with the same primary amino acid sequence as an amino acid sequence according to claim 117; and
b) forming a disulphide bridge in said amino acid sequence so as to provide the amino acid sequence.

150. Method for preparing an amino acid sequence, which method at least comprises the step of:
a) expressing a nucleotide sequence or nucleic acid according to claim 147, so as to provide an amino acid sequence;
and optionally further comprising:
b) isolating the amino acid sequence obtained in step b);
and:
c) forming a disulphide bridge in the amino acid sequence obtained in step a) or, when step b) is performed, in the amino acid sequence obtained in step b), respectively, so as to provide the amino acid sequence.

151. Amino acid sequence, compound or construct, obtained via the method of claim 41.

152. Pharmaceutical composition that comprises at least one amino acid sequence according to claim 117; and optionally at least one pharmaceutically acceptable carrier, diluent or excipient.

153. Method for preparing a compound or construct, said method at least comprising the step of linking at least one amino acid sequence according to claim 1 to at least one therapeutic moiety, optionally via one or more suitable linkers or spacers.

154. Method for preparing a compound or construct, said method at least comprising the step of linking an amino acid sequence according to claim 25 to at least one therapeutic moiety, optionally via one or more suitable linkers or spacers.

155. Method for preparing a compound or construct, said method at least comprising the step of linking at least one amino acid sequence according to claim 117 to at least one therapeutic moiety, optionally via one or more suitable linkers or spacers.

156. Compound or construct, obtained via the method of claim 153.

157. Pharmaceutical composition that comprises at least one amino acid sequence according to claim 156; and optionally at least one pharmaceutically acceptable carrier, diluent or excipient.


SEQUENCE LISTING (32 sequences)

SEQ ID NO: 1 - protein, 34 aa, Lama glama
DTAVYYCNAAASYSDYDVFGGGTDFGPWGQGTQV

SEQ ID NO: 2 - DNA, 102 bp, Lama glama
gacacggccgtttattattgtaatgcagccgcctcctatagcgactatgacgtctttgggggaggaactgactttggtccctggggccaagggacccaggtc

SEQ ID NO: 3 - protein, 18 aa, Lama glama
AASYSDYDVFGGGTDFGP

SEQ ID NO: 4 - DNA, 21 bp, Artificial
  primer ABL051
ggctgagctgggtggtcctgg

SEQ ID NO: 5 - DNA, 21 bp, Artificial
  primer ABL0052
ggctgagtttggtggtcctgg

SEQ ID NO: 6 - DNA, 23 bp, Artificial
  primer ABL003
ggtacgtgctgttgaactgttcc

SEQ ID NO: 7 - DNA, 53 bp, Artificial
  primer For1Sfi
gtcctcgcaactgcggcccagccggccatggcggacacggccgbctattactg

SEQ ID NO: 8 - DNA, 53 bp, Artificial
  primer For2Sfi
gtcctcgcaactgcggcccagccggccatggcggacacggccgtttatwactg

SEQ ID NO: 9 - DNA, 53 bp, Artificial
  primer For3Sfi
gtcctcgcaactgcggcccagccggccatggcggacacggccgtgtattaytg

SEQ ID NO: 10 - DNA, 53 bp, Artificial
  primer For4Sfi
gtcctcgcaactgcggcccagccggccatggcggacacggccgtctattwttg

SEQ ID NO: 11 - DNA, 53 bp, Artificial
  primer For5Sfi
gtcctcgcaactgcggcccagccggccatggcggacacggccgwttattattg

SEQ ID NO: 12 - DNA, 53 bp, Artificial
  primer For6Sfi
gtcctcgcaactgcggcccagccggccatggcggacacggccatytattwctg

SEQ ID NO: 13 - DNA, 53 bp, Artificial
  primer For7Sfi
gtcctcgcaactgcggcccagccggccatggcggacacgggactytattactg

SEQ ID NO: 14 - DNA, 56 bp, Artificial
  primer Back1Not
gagtcattctcgacttgcggccgctgaaccgcctccgacctgrgtbccctggcccc

SEQ ID NO: 15 - DNA, 56 bp, Artificial
  primer Back2Not
gagtcattctcgacttgcggccgctgaaccgcctccgacctkggtcccttkgcccc

SEQ ID NO: 16 - DNA, 56 bp, Artificial
  primer Back3Not
gagtcattctcgacttgcggccgctgaaccgcctccgacctgggtccccggsccyc

SEQ ID NO: 17 - DNA, 56 bp, Artificial
  primer Back4Not
gagtcattctcgacttgcggccgctgaaccgcctccgacctgggtcccctghcccc

SEQ ID NO: 18 - DNA, 56 bp, Artificial
  primer Back5Not
gagtcattctcgacttgcggccgctgaaccgcctccgacctgggtcccctggccgt

SEQ ID NO: 19 - DNA, 57 bp, Artificial
  primer Back1cysRNot
gagtcattctcgacttgcggccgctgaaccggctccgacctgrgtbccctggcacct

SEQ ID NO: 20 - DNA, 57 bp, Artificial
  primer Back1cysWNot
gagtcattctcgacttgcggccgctgaaccggctccgacctgrgtbccctggcacca

SEQ ID NO: 21 - DNA, 57 bp, Artificial
  primer Back2cysWNot
gagtcattctcgacttgcggccgctgaaccggctccgacctkggtcccttkgcacca

SEQ ID NO: 22 - DNA, 57 bp, Artificial
  primer Back3cysWNot
gagtcattctcgacttgcggccgctgaaccggctccgacctgggtccccgggcacca

SEQ ID NO: 23 - DNA, 57 bp, Artificial
  primer Back3cysRNot
gagtcattctcgacttgcggccgctgaaccggctccgacctgggtccccgggcatct

SEQ ID NO: 24 - DNA, 57 bp, Artificial
  primer Back4cysWNot
gagtcattctcgacttgcggccgctgaaccggctccgacctgggtcccctggcacca

SEQ ID NO: 25 - DNA, 57 bp, Artificial
  primer Back5cysWNot
gagtcattctcgacttgcggccgctgaaccggctccgacctgggtcccctggcagta

SEQ ID NO: 26 - protein, 20 aa, Artificial
  non-constrained truncated 17D12 peptide
AAASYSDYDVFGGGTDFGPA

SEQ ID NO: 27 - protein, 22 aa, Artificial
  constrained truncated 17D12 peptide
CAAASYSDYDVFGGGTDFGPAC

SEQ ID NO: 28 - protein, 19 aa, Artificial
  truncated 17D12 peptide
AAASYSDYDVFGGGTDFGP

SEQ ID NO: 29 - protein, 20 aa, Artificial
  truncated 17D12 peptide
CAAASYSDYDVFGGGTDFGP

SEQ ID NO: 30 - protein, 162 aa, Artificial
  2D3-17D12 fusion protein
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSDTAVYYCNAAASYSDYDVFGGGTDFGPWGQGTQVGGGS

SEQ ID NO: 31 - protein, 34 aa, Artificial
  Cys residue in FR3 of SEQ ID NO1 replaced with a Ser
DTAVYYSNAAASYSDYDVFGGGTDFGPWGQGTQV

SEQ ID NO: 32 - protein, 43 aa, Artificial
  Cys residue in FR3 of SEQ ID NO1 replaced with a Ser; Gly4Ser-Gly3Ser linker added at N-terminus
GGGGSGGGSDTAVYYSNAAASYSDYDVFGGGTDFGPWGQGTQV